(19) United States
(12) Patent Application Publication
(10) Pub. No.: US 2024/0368650 A1
(43) Pub. Date: Nov. 7, 2024

(54) POLYPEPTIDES WITH D-PSICOSE 3-EPIMERASE ACTIVITY

(71) Applicant: AMBROSIA BIO LTD., Jerusalem (IL)

(72) Inventors: Ziv Zeev ZWIGHAFT, Tel Aviv (IL); Dan S. TAWFIK, Karmi Yosef (IL)

(73) Assignee: AMBROSIA BIO LTD., Jerusalem (IL)

(21) Appl. No.: 18/574,397

(22) PCT Filed: Jun. 27, 2022
(86) PCT No.: PCT/IL2022/050690

§ 371 Date: Dec. 27, 2023

Related U.S. Application Data
(60) Provisional application No. 63/215,472, filed on Jun. 27, 2021.

(51) Int. Cl. C12P 19/02; C12N 9/90

(52) U.S. Cl. CPC C12P 19/02; C12N 9/90; C12Y 501/03

(57) ABSTRACT

The present invention provides polypeptides having 3-epimerase activity, such as D-psicose 3-epimerase activity, polynucleotides encoding same, and transgenic cells including same. Further provided is a method for producing D-psicose.

### CROSS REFERENCE TO RELATED APPLICATIONS

This application claims the benefit of priority to U.S. Provisional Patent Application No. 63/215,472 filed Jun. 27, 2021, entitled “POLYPEPTIDES WITH D-PSICOSE 3-EPIMERASE ACTIVITY” the contents of which are incorporated herein by reference in their entirety.

### FIELD OF INVENTION

The present invention relates to isolated polypeptides having 3-epimerase activity, polynucleotides encoding same, and method of using same.

### BACKGROUND

The excessive consumption of conventional sugars such as sucrose, can have an impact on human health. There is a need for alternatives to lower sugar intake. Rare sugars such as monosaccharides, existing in nature in limited quantities, can be used as such alternatives. Rare sugars are useful additives and compounds in foods and pharmaceuticals and the beneficial metabolic health effects of rare sugars have been shown in human trails, animal experiments and in in vitro experiments. The chemical synthesis of rare sugars suffers from several disadvantages including immoderate reaction conditions, generation of excessive chemical waste, the formation of undesired byproducts, and difficult purification process. In contrast, the biosynthesis of rare sugars by enzymatic pathways is more environmentally friendly, have moderate reaction conditions and high specificity. Enzymes that catalyze the epimerization of ketoses and ketose derivatives play an important role in the biosynthesis of rare sugars. Of the rare sugars, D-psicose (D-ribo-2-hexulose or D-allulose) is the one most intensively studied as a healthier substitute for sucrose, having 70% of the relative sweetness but only 0.3% of the energy of sucrose and high solubility. This sugar has recently received a generally recognized as safe (GRAS) status from the U.S. Food and Drug Administration (FDA) and applications for its approval as a food ingredient in the European Union are under investigations.

Enzymes from the D-tagatose-epimerase (DTE) and D-psicose 3-epimerase (DPE, also known as D-allulose 3-epimerase (DAE)), families catalyze the reversible conversion of D-fructose to D-psicose at the C3 position. In addition, L-ribulose 3-epimerase (LRE) is also known to convert D-fructose to D-allulose. Optimal catalytic efficacy of these known 3-epimerases, require ideal pH and temperature ranges (pH between 7.0 and 8.0 and temperatures between 55° C. and 70° C.). However, these optimal ranges may result in the degradation of both D-fructose and D-allulose. Stable D-fructose and D-psicose at these temperature ranges require low pH conditions and an enzyme capable of operating in an acidic environment. Such enzymes may be found in acidophilic organisms.

Acidophiles are a diverse group of ecologically and economically important organisms, which thrive in acidic natural and artificial man-made environments. They possess a network of cellular adaptations that regulate pH inside the cell. Several extracellular enzymes from acidophiles are functional at much lower pH than the cytoplasmic pH. Enzymes like amylases, proteases, ligases, cellulases, xylanases, α-glucosidases, endoglucanases, and esterases stable at low pH are known from various acidophilic microbes. However, enzymes capable of catalytic reaction at low pH conditions are rare. There is a need for finding novel sources of acid-stable enzymes. One such source may be found in the ancestors of present-day, extant, enzymes of acidophiles.

Naturally occurring enzymes exhibit high rates of catalytic activity, are enantiomerically pure catalysts responsible for the conservation of chirality providing enantioselectivity in enzymatic catalysis, and due to their biodegradability, have low environmental impact. Despite these advantages, the availability of enzymes for catalyzing reactions is limited, as enzymes with required properties are not always found in nature.

Ancestral sequence reconstruction (ASR) infers ancestral amino acid sequences using homologues amino acids sequences input. ASR provides an information tool for amino acids substitutions remote from the active site, which can influence activity and stability. Enzyme design using this method comprise inference of an ancestral sequence based on a comparison of homologues amino acid sequences available in public databases, artificial synthesis of a gene encoding the inferred amino acid sequence, and expression of the gene in a suitable host organism. In combination with empirical analysis, this method was used to define various physical properties of the ancestral proteins, including thermostability and substrate specificity, and to identify key amino acids residues. Ancestral design creates enzymes with desired traits that are not found in nature and can improve natural enzymes by substitution of putative ancestral amino acids into natural enzymes without compromising the catalytic activity. This have led to the use of ASR in the engineering of proteins and enzymes that are potentially useful in industrial process.

There is still a great need for novel polypeptides having 3-epimerase activity, that are capable of converting D-fructose to D-psicose at low pH conditions.

### SUMMARY

In one aspect, there is provided a polypeptide comprising an amino acid sequence selected from the group consisting of:
(i) SEQ ID Nos.: 1-12 and 14-16, or a functional analog having at least 88% sequence homology thereto; and
(ii) SEQ ID No. 13, or a functional analog having at least 92% sequence homology thereto.

In one embodiment, the polypeptide is characterized by having a 3-epimerase activity.

In one embodiment, the 3-epimerase activity comprises D-psicose 3-epimerase activity.

In one embodiment, the peptide is an isolated polypeptide or a synthetic polypeptide.

In another aspect, there is provided a polynucleotide encoding the polypeptide of the invention.

In one embodiment, the polynucleotide comprising a nucleic acid sequence selected from the group consisting of: SEQ ID Nos.: 17-32.

In one embodiment, the polynucleotide is an isolated or an artificial polynucleotide.

In another aspect, there is provided a plasmid or an expression vector comprising the polynucleotide of the invention.

In another aspect, there is provided a transgenic, transformed, or a transfected cell comprising: the polypeptide of the invention; the polynucleotide of the invention; the plasmid or expression vector of the invention; or any combination of (a) to (c).

In one embodiment, the transgenic cell or said transfected cell is selected from the group consisting of: Bacillus licheniformis, Saccharomyces cerevisiae, Schizosaccharomyces pombe, Pseudomonas putida, Pichia sp., Aspergillus sp., Trichoderma reesei, Corynebacterium glutamicum, E. coli and B. subtilis.

In another aspect, there is provided an extract derived from the transgenic cell or the transfected cell of the invention, or any fraction thereof.

In one embodiment, the extract comprising said polynucleotide, said polypeptide, or both.

In another aspect, there is provided a transgenic plant, a transgenic plant tissue or a plant part, comprising: the polypeptide of the invention; the polynucleotide of the invention; the plasmid or expression vector of the invention; the transgenic cell or transfected cell of the invention; or any combination of (a) to (d).

In another aspect, there is provided a composition comprising:
a. the polypeptide of the invention
b. the polynucleotide of the invention;
c. the plasmid or expression vector of the invention;
d. the transgenic cell or transfected cell of the invention;
e. the extract of the invention;
f. the transgenic plant tissue or plant part of the invention; or
g. any combination of (a) to (f),
   and an acceptable carrier.

In one embodiment, the composition further comprises a divalent metal cation.

In one embodiment, the divalent cation is selected from the group consisting of: Co²⁺, Mg²⁺, Mn²⁺, Mo²⁺, Ni², and any combination thereof.

In one embodiment, the composition further comprising an aqueous solvent.

In one embodiment, the polypeptide is bound to a solid support.

In one embodiment, bound is via a covalent bond or a non-covalent interaction.

In one embodiment, the composition further comprises a chemical agent capable of binding both said polypeptide and said solid support.

In one embodiment, the composition further comprising fructose, D-psicose, or both.

In another aspect, there is provided a method for synthesizing a polypeptide having 3-epimerase activity, comprising the steps: providing a cell comprising a plasmid or an expression vector comprising a nucleic acid sequence having at least 70% sequence identity to a nucleic acid sequence selected from the group consisting of: SEQ ID Nos.: 17-32; and culturing said cell from step (a) such that a polypeptide encoded by said plasmid or an expression vector is expressed, thereby synthesizing the polypeptide.

In one embodiment, the method further comprising at least one step selected from the group consisting of: recovering said polypeptide, at least partially purifying the polypeptide, immobilizing said polypeptide, and any combination thereof.

In one embodiment, the cell is a prokaryote cell or a eukaryote cell.

In one embodiment, the cell is a transgenic cell or a cell transfected with the plasmid or expression vector of the invention.

In one embodiment, the method comprising a step preceding step (a), comprising introducing or transfecting said cell with said plasmid or expression vector.

In another aspect, there is provided a method for producing D-psicose comprising the steps: contacting the composition of the invention with an effective amount of fructose, thereby obtaining a reaction mixture; and subjecting said reaction mixture to conditions suitable for at least partial conversion of said fructose to D-psicose, thereby producing D-psicose.

In one embodiment, the conditions comprise: (i) a temperature between 25° C. and 75° C.; (ii) a pH of between 3 and 9, or both (i) and (ii).

In one embodiment, subjecting comprises contacting said reaction mixture with a divalent metal cation.

In one embodiment, the divalent cation is selected from the group consisting of: Co2+, Mg2+, Mn2+, Mo2+, Ni2, and any combination thereof.

In one embodiment, the divalent metal cation is present in said reaction mixture in a concentration ranging from 0.1 mM to 10 mM.

In one embodiment, the method further comprising a step comprising isolating said D-psicose from said reaction mixture.

In one embodiment, isolating comprises: extraction, precipitation, membrane filtration, or any combination thereof.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

FIG. 1 includes a chromatogram of Ni-sepharose FF Column separation of C3_N58 (corresponding to SEQ ID No.: 11). The solid line (marked by an arrow) indicates absorbance at 280 nm. Numerals 1-5 depict different stages of separation: 1-Loading of the sample; 2-Wash; 3-Wash with buffer containing 25 mM imidazole; 4-Elution with buffer containing 150 mM imidazole buffer; 5-Strip.

FIG. 2 includes a chromatogram of Ni-sepharose FF Column separation of C3_N72 (corresponding to SEQ ID No.: 13). Separation was performed as previously described in FIG. 1.

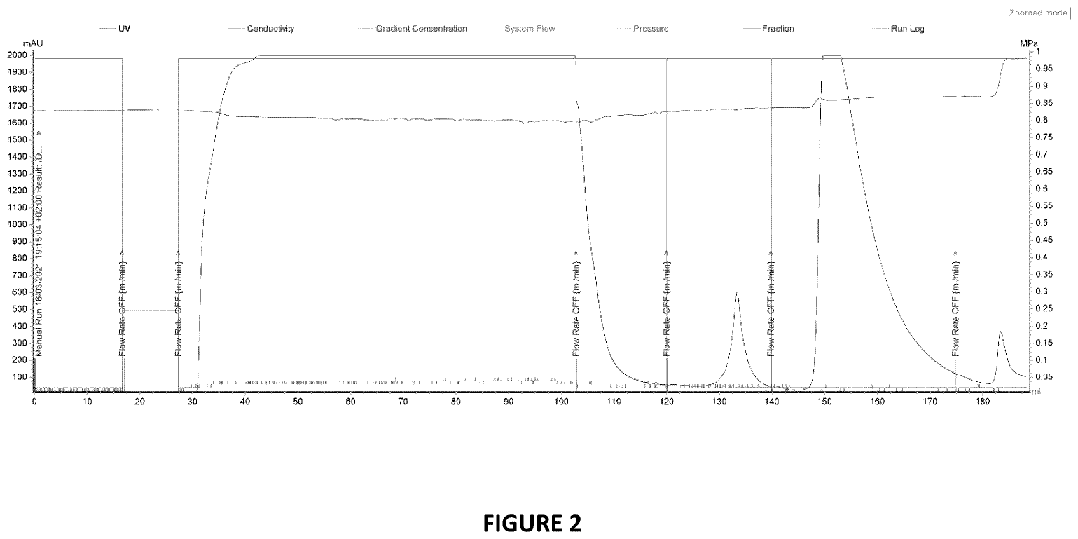

FIG. 3 includes a chromatogram of Ni-sepharose FF Column separation of C4_N116 (corresponding to SEQ ID No.: 16). Separation was performed as previously described in FIG. 1.

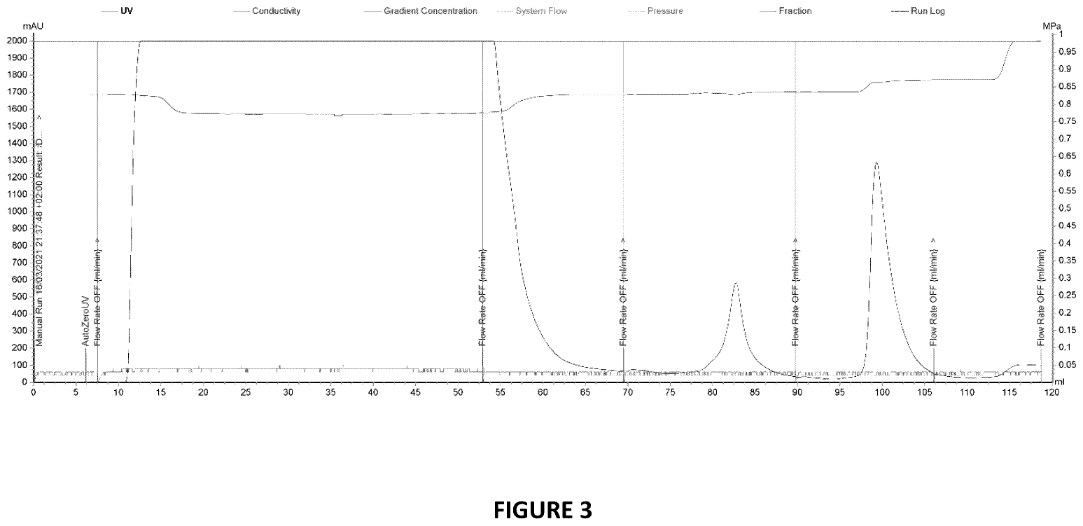

FIG. 4 includes a micrograph of a Coomassie stain of an SDS-PAGE of the C3N58, C3N72, and C4N116 polypeptides (corresponding to SEQ ID Nos.: 11, 13, and 16, respectively).

FIG. 5 includes HPLC (high performance liquid chromatography) chromatograms of crude reaction mixture containing an aqueous solution of D-fructose (as a substrate), and the C3_N72 polypeptide (corresponding to SEQ ID No.: 13) under neutral conditions (pH 7). The aliquots have been sampled at different reaction times (0, 5, 10, 15, 30, 45, 60 and 120 min). The HPLC chromatograms demonstrate conversion of D-fructose (peak at 7.6 min) into allulose (at 6.3 min).

FIG. 6 includes HPLC (high performance liquid chromatography) chromatograms of crude reaction mixture containing an aqueous solution of D-fructose (as a substrate), and the C3_N72 polypeptide (corresponding to SEQ ID No.: 13) under moderate acidic conditions (pH 6). The aliquots have been sampled at different reaction times (0, 5, 10, 15, 30, 45, 60 and 120 min). The HPLC chromatograms demonstrate conversion of D-fructose (peak at 7.6 min) into allulose (at 6.3 min).

FIG. 7 presents graphs showing catalytic activity of the exemplary polypeptides of the invention, as assessed in a pilot-scale experiment. Lot A refers to SEQ ID No.: 11, Lot B refers to SEQ ID No.: 13.

FIGS. 8A-B present graphs showing catalytic activity of the exemplary polypeptide of the invention (SEQ ID No.: 12, assigned as N68cons) over time, compared to a commercial control (DT3E from P.Cichorri) tested at pH=6 (FIG. 8B), and at pH=7 (FIG. 8A).

### DETAILED DESCRIPTION

The present invention, in some embodiments, is directed to polypeptides having 3-epimerase activity, polynucleotides encoding same, and methods of using same.

According to some embodiments, there is provided a polypeptide encoded by: (a) the polynucleotide disclosed herein; or (b) the plasmid or expression vector disclosed herein.

In some embodiments, the polypeptide comprises an amino acid sequence selected from SEQ ID Nos.: 1-16, or a functional analog comprising at least 88% homology thereto. In some embodiments, the polypeptide of the invention comprises an amino acid sequence selected from the group consisting of: (i) SEQ ID Nos.: 1-12 and 14-16, or a functional analog having at least 88% sequence homology thereto; and (ii) SEQ ID No. 13, or a functional analog having at least 92% sequence homology thereto. In some embodiments, the polypeptide of the invention comprises an amino acid sequence selected from the group consisting of SEQ ID Nos.: 1-16 or a functional analog having at least 88%, at least 89%, at least 90%, at least 95%, at least 97%, at least 99%, or 100% sequence homology thereto including any range between.

In some embodiments, the polypeptide of the invention comprises an amino acid sequence selected from SEQ ID Nos.: 4, 9, 10, 11, 12, 13, 14, and 16 or a functional analog having at least 88%, at least 89%, at least 90%, at least 92%, at least 93%, at least 94%, at least 95%, at least 96%, at least 97%, at least 98%, at least 99%, or 100% sequence homology thereto, including any range between. In some embodiments, the polypeptide of the invention comprises an amino acid sequence selected from SEQ ID Nos.: 11, 12, and 16 or a functional analog having at least 88%, at least 89%, at least 90%, at least 92%, at least 93%, at least 94%, at least 95%, at least 96%, at least 97%, at least 98%, at least 99%, or 100% sequence homology thereto, including any range between. In some embodiments, the polypeptide of the invention comprises an amino acid sequence selected from SEQ ID Nos.: 11, 12, 13, and 16 or a functional analog having at least 92%, at least 93%, at least 94%, at least 95%, at least 96%, at least 97%, at least 98%, at least 99%, or 100% sequence homology thereto, including any range between.

In some embodiments, the polypeptide of the invention comprises an amino acid sequence selected from SEQ ID Nos.: 4, 9, 10, 11, 12, 13, 14, and 16 or a functional analog having at least 88%, at least 89%, at least 90%, at least 92%, at least 93%, at least 94%, at least 95%, at least 96%, at least 97%, at least 98%, at least 99%, or 100% sequence homology thereto, including any range between; wherein the polypeptide of the invention is in a form of a homo tetramer, or a homo octamer.

In some embodiments, the polypeptide of the invention is in a form of a homo tetramer, or a homo octamer; wherein the polypeptide comprises an amino acid sequence selected from SEQ ID Nos.: 11, 12, 13 and 16 or a functional analog having at least 88%, at least 89%, at least 90%, at least 92%, at least 93%, at least 94%, at least 95%, at least 96%, at least 97%, at least 98%, at least 99%, or 100% sequence homology thereto, including any range between.

In some embodiments, the polypeptide of the invention comprises an amino acid sequence of SEQ ID No. 13 or a functional analog having at least 92%, at least 93%, at least 94%, at least 95%, at least 96%, at least 97%, at least 98%, at least 99%, at least 100%, sequence homology thereto including any range between.

In some embodiments, the functional analog comprises an amino acid sequence with at least 80%, at least 85%, at least 88%, at least 89%, at least 90%, at least 92%, at least 95%, at least 97%, at least 99%, or 100% homology or identity to any one of SEQ ID Nos.: 1-16. Each possibility represents a separate embodiment of the invention.

As used herein, the term functional analog refers to any polypeptide characterized by having the 3-epimerase activity of the polypeptide of the invention as described herein and having sequence homology thereto, as described herein. In some embodiments, the term functional analog refers to any polypeptide characterized by having the 3-epimerase activity of the polypeptide of the invention as described herein and having at least 88%, or at least 92% sequence homology thereto.

In some embodiments, the polypeptide of the invention comprises an amino acid sequence selected from SEQ ID Nos: 1-16, or a functional analog comprising at least 70% at least 75%, at least 80%, at least 85%, at least 88% homology thereto, including any range between, wherein the polypeptide is an oligomer comprising at least 3, at least 4, at least 5, at least 6, at least 8, polypeptide monomers. In some embodiments, the polypeptide of the invention is an oligomer comprising between 4 and 8, between 4 and 6, between 6 and 8 or more polypeptide monomers. In some embodiments, each of the polypeptide monomers comprises an amino acid sequence selected from SEQ ID Nos: 1-16. In some embodiments, each of the polypeptide monomers comprises the same amino acid sequence. In some embodiments, at least one of the polypeptide monomers comprises a different amino acid sequence.

In some embodiments, the polypeptide of the invention is in a form of a homo tetramer. In some embodiments, the polypeptide of the invention is in a form of a homo octamer. In some embodiments, the polypeptide monomers are bound to each other via non-covalent bonds or non-covalent interactions. In some embodiments, the polypeptide monomers are bound to each other via electrostatic interactions.

Without being bound to any particular theory or mechanism, it is postulated that the electrostatic interaction between E-195 and K-196 of the amino acid sequence is responsible for the formation of the oligomer, wherein the oligomer is as described herein.

The term “analog” as used herein, refers to a polypeptide that is similar, but not identical, to the polypeptide of the invention that still is capable of binding fructose or still comprises the fructose-binding pocket. An analog may have deletions or mutations that result in an amino acids sequence that is different than the amino acid sequence of the polypeptide of the invention. It should be understood that all analogs of the polypeptide of the invention would still be capable of binding fructose or still comprise the fructose binding pocket. Further, an analog may be analogous to a fragment of the polypeptide of the invention, however, in such a case the fragment must comprise at least 50 consecutive amino acids of the polypeptide of the invention.

As used herein, the term “analog” includes any peptide having an amino acid sequence substantially identical to one of the sequences specifically shown herein in which one or more residues have been conservatively substituted with a functionally similar residue and which displays the abilities as described herein. Examples of conservative substitutions include the substitution of one non-polar (hydrophobic) residue such as isoleucine, valine, leucine or methionine for another, the substitution of one polar (hydrophilic) residue for another such as between arginine and lysine, between glutamine and asparagine, between glycine and serine, the substitution of one basic residue such as lysine, arginine or histidine for another, or the substitution of one acidic residue, such as aspartic acid or glutamic acid for another. Each possibility represents a separate embodiment of the present invention.

In some embodiments, the polypeptide is an isolated polypeptide or a synthetic polypeptide.

As used herein, the terms “peptide”, “polypeptide” and “protein” are interchangeable and refer to a polymer of amino acid residues. In another embodiment, the terms “peptide”, “polypeptide” and “protein” as used herein encompass native peptides, peptidomimetics (typically including non-peptide bonds or other synthetic modifications) and the peptide analogues peptoids and semipeptoids or any combination thereof. In another embodiment, the peptides, polypeptides and proteins described have modifications rendering them more stable while in the organism or more capable of penetrating into cells. In one embodiment, the terms “peptide”, “polypeptide” and “protein” apply to naturally occurring amino acid polymers. In another embodiment, the terms “peptide”, “polypeptide” and “protein” apply to amino acid polymers in which one or more amino acid residue is an artificial chemical analogue of a corresponding naturally occurring amino acid.

As used herein, the terms “isolated protein” refers to a protein that is essentially free from contaminating cellular components, such as carbohydrate, lipid, or other proteinaceous impurities associated with the nucleic acid in nature. Typically, a preparation of an isolated protein contains the protein in a highly purified form, e.g., at least about 80% pure, at least about 90% pure, at least about 95% pure, greater than 95% pure, or greater than 99% pure. In some embodiments, the isolated protein is a synthesized protein. Synthesis of protein is well known in the art and may be performed, for example, by heterologous expression in a transformed cell, such as exemplified herein.

In some embodiments, the polypeptide comprises or consists of the amino acid sequence:

(SEQ ID NO: 1)
MKFAICNETFQPDAPPLWPLEKAFAYARELGYTGIEFAPFDATDVSAER
RAEVRELLEKYQLESIGLHWLLAKTELTSPDAAVRDRTSEYLAELARLC
RDLGGSVMCGVLGSPQQRNLLPGVTHDQAMEYAADVIRAAMPALEDHGV
TLALEPLGPAEGDFLLTAESGIQLAQMIDSPHVRLHLDVKAMSLEAKPI
PDIIRDSADYLAHFHANDPNKRGPGMGDVDFVPIFQALQEINYQGWVSV
EVFDYEPGVVERLARESISYMQRCLDKLYSSVSGGERPPPDN.

In some embodiments, the polypeptide comprises or consists of the amino acid sequence:

(SEQ ID NO: 2)
MKFAICQELFEPDAPPLTDWAEQCRIIAEVGYTGIEVAPFDLASVPAAT
LTEMRNTAERHGLEIIGLHWLLAKTNLTSPDAAVRTATADYLKLLADTC
AALGGRVLAGVFGSPQQRNLLAGVTTEQAMDFAAEVFRAAMPTFAERDV
VLCMEPLTPKETDFINTCADAVALMQMVDHPSFVLHQDVKAMLLAESDV
PELIHRHKDICGHFHVNDTNLLGPGMGETDYHPILKALLEVGYDGWVSV
EVFDYSPGAVEKIAKKSMDYMQQVLSDLYKAVSGVSGPPCDN.

In some embodiments, the polypeptide comprises or consists of the amino acid sequence:

(SEQ ID NO: 3)
MKLGIHSFVWTGGSTQEDLEGAMEKSHELGYDLIEFPRLDPKKFDVAWLA
KRLEDSGLKVVVTMGLPLDGDISSEDADVVKSGEQILDDAVAVTRDLGGE
KLGGILFSAHGKYQSMPTRKGWDNSVAVMRKVADRAKAAGVTLNLEIVNR
FESNLLNTTAQGLAFIEDTGSDNIFLHLDTFHMNIEEADPAQAIRHAGDK
LGYFHIGESNRGYLGAGTIDFPPIFDALVAIGYEDYVTFESFSSEVVDKD
LSITTGIWRNTWTDNVEVARHAKAFIEARYGEARRKAQIA.

In some embodiments, the polypeptide comprises or consists of the amino acid sequence:

(SEQ ID NO: 4)
MRLGIHSFVWTGGQTQEGLEMALEKSAEHGYRLIEFAYLRPEKFDLDRLA
KKAQSLDLEIAVTMGLPLDADVSSEDADVVARGKAMLADAVKAVRDIGGN
KLGGILYSAHTKYNRMPTQRGWKNSVEAIAKTAEIAKACGVDLVLEVVNR
FETNLLNTTAQGLKFIEDVGSDDVRLHLDTFHMNIEEADPAAAIRLAGDK
LGYFHIGENNRGYLGDGVIDFDAIFDALLDIGYDRDIVFESFSSAVVDEG
LSLACAIWRDTWTDNDELAAHAKQFIELKYDEAQRRRATN.

In some embodiments, the polypeptide comprises or consists of the amino acid sequence:

(SEQ ID NO: 5)
MRLGIHSFVWTGGQTQSMLEEAMEKSASCGYNLIEFAYLRPEKFDLDALA
KRAQALKLDIAVTMGLPFSHDVSSEDADTVKAGEGMLADAVRAVRDIGGS
KLGGILYSAHSKYSRMPTKKGWDNSVAAIAKTADLAKSAGVDLVLEVVNR
FETNLLNTTAQGLAFIEQVGSEHVRLHLDTFHMNIEEANVAAAIRLAGDK
IGYFHIGESNRGYLGDGNINFDLIFDALLDIDYQRDITFESFSGAVVDEG
LSLACAIWRDTWTENMPLAQHAKQFIDLKMDEAKRRRATV.

In some embodiments, the polypeptide comprises or consists of the amino acid sequence:

(SEQ ID NO: 6)
MRLGIHSFVWTGGQTQEGLEMALEKSAEHGYRCIEFAYLRPELFDLDRLA
RKSQSLDVQIGVTMGLPLDKDVSSEDPDVVAAGKQMLADAVRAVRDVGGD
KLGGILYSAHTKYNRQPTADGWKNSVEAIAETGEIAKAAGVDLVLEVVNR
FETNLLNTTAQGLKFIDDTGSDHVRLHLDTFHMQIEEANPAAAIRLAGDK
LGYFHIGESNRGYLGDGTIDWDPIFDALLDIGYDRDVVFESFSTAIVDEG
LSLACAIWRDTWTENDELAAHAKQFIELKWAEAQRRRATN.

In some embodiments, the polypeptide comprises or consists of the amino acid

(SEQ ID NO: 7)
MRLGIHSFVWTGGQTQEGLEMALEKSAEHGYRTIEFAYLRPELFDLDRLA
RKAQSLDVEIGVTMGLPLDKDVSSEDRDVVARGKQMLADAVRAVRDIGGN
KLGGILYSAHTKYNRLPTADGWKNSVEAIAETGEIAKAAGVDLVLEVVNR
FETNLLNTTAQGLKFIDDVGSDHVRLHLDTFHMNIEEANPAAAIRLAGDK
LGYFHIGESNRGYLGDGNIDFDLIFDALLDIDYQRDVVFESFSTAIVDEG
LSLACAIWRDTWTENDPLAAHAKQFIELKYAEAQRRRATN.

In some embodiments, the polypeptide comprises or consists of the amino acid sequence:

(SEQ ID NO: 8)
MRLGIHSFVWTGGQTQEGLEMALNKTAEHGYRTIEFAYLRPEKFNLDRLA
KLAQSLDVEIGVTMGLPLDKDVSSEDAAAVAAGKQMLADAVRAVRDIGGN
KLGGILYSAHTKYNRQPTKKGWDNSVAAIAATAEIAKEAGVDLVLEVVNR
FETNLLNTTAQGLKFISEVGSDHVRLHLDTFHMNIEEANPAAAIRLAGDK
IGYFHIGESNRGYLGDGVINFDLIFDALTDIDYKRDIVFESFSTAVVDEG
LSLACAIWRDTWTDNDELAAHAKRYIELKYDEAKRRRATN.

In some embodiments, the polypeptide comprises or consists of the amino acid sequence:

(SEQ ID NO: 9)
MKLGIHSFVWTGGTTQEDLEGAMEKSHDLGYGLIEFPRLDPKKFDVSWLA
KRLEDYGLKVVVTMGLPLDGDISSEDAAVVKRGEQILDDAVAITRDLGGE
KLGGIIFSAHTKYQSMPTRKGWDNSVAALSRVADRAKAAGVSLNLEIVNR
FESNLLNTTAQGLAFIEETGSDNIFLHLDTFHMNIEEADPAQAIRLAGDK
LGYFHIGESNRGYLGSGVINFPAIFDALVAIGYEDWITFESFSSEVVDED
LSITCAIWRNTWTDNVEVARLAKAFIEARYGEARRKAQTA.

In some embodiments, the polypeptide comprises or consists of the amino acid sequence:

(SEQ ID NO: 10)
MQIGAHGLVFTGTFDEAGLRRAIEGTKHAGFDLIEIPLMDVDGFDSALAG
RMLADNDLAVTASLGLTAATDLTSEDPAVVAAGERMLERCLDHVAAMGGD
VLCGVIYSAMQKYMAPATERGIANSQAAIARLAEKAAERGIRLSLEVVNR
YESNVENTGRGALAFLDGVGRDDVSVHLDTYHMNIEESDLFQPVHDVAGR
LGYVHIGESHRGYLGTGTVDFGTFFRALHDVRYDGPVVFESFSSAVVSAE
LSNTLGIWRNLWEDSDDLAAHANRFIRDGLHAVRTIDL.

In some embodiments, the polypeptide comprises or consists of the amino acid sequence:

(SEQ ID NO: 11)
MDIGCHGLVWTGSFDAQGIRLAAQKTKEAGFDLIEYPLMDPFTFDTEAAV
SALAEHGLKASASLGLSDSTDVTSTDPEVVAAGEALLLRAVDVLADMGGT
HLCGVIYSAMKKYMEPVTAEGLENSKRTIGRVADHAADRGVNVSLEVVNR
YETNVLNTARQAIRFVADVDRPNLGIHLDTYHMNIEESDMFAPVLDAGEV
LHYVHIGESHRGYLGTGSVDFDNFFKALGRIGYDGPITFESFSSAVVAPD
LSRMLGIWRNLWTDNADLGAHANRFIRDKLTAVETISL.

In some embodiments, the polypeptide comprises or consists of the amino acid sequence:

(SEQ ID NO: 12)
MEYGGHALVWAGDWSPEGARQAISGAARAGYDYIEIALLDPWKVDVAMTK
DLLQEYGLRAHASLGLSPTTDVTSTDPAIVAKGDELLRKATDVLHALGGT
ELCGVIYCALGKYPGPASKENRANSVAAMQRLADYAADKGININLEVVNR
YETNIMNTGIEGLKFLEEVNRPNAYLHLDTYHMNIEEDGMEKAVLAAGDR
LGYVHIGESHRGYLGSGNVDFDTFFAALKKINYQGPITFESFSSAVVDPA
LSNALCVWRNLWSDSDDLAKKALKFMKARYSGNSQRSP.

In some embodiments, the polypeptide comprises or consists of the amino acid sequence:

(SEQ ID NO: 13)
MEYGGHALVWSGDWTPEGARKAISGAARAGYDYIEIALLDPWKVDVALTK
DLLQEYNLRAHASLGLSAATDVTSTDPAIVAKGDELLRKATDVLHAIGGT
ELCGVIYCALGKYPGPASKENRANSVAAMQRLADYAADKGININLEVVNR
YETNIMNTGLEGLAFLDEVNRPNAYLHLDTYHMNIEEDGMEKSVLAAKDR
LGYVHIGESHRGYLGTGNVDFDSFFAALKKIDYQGPITFESFSSEVVDPK
LSNTLCVWRNLWHDSDDLAKKALRFMKERYPGNPQRSP.

In some embodiments, the polypeptide comprises or consists of the amino acid sequence:

(SEQ ID NO: 14)
MKYGGHALVWSGDWTPEGARQAISGAARAGYDYIEIALLDPWKVDVALTK
DLLQEFNLRAHASLGLSAATDVTSTDMAIVAKGDELLRKATDVLAAIGGK
ELCGVIYCALGKYPGPASKENRNNSVAAMQRLADYAADKDININLEVVNR
YETNIINTGIEGLAFLDEVDRPNAYLHLDTYHMNIEEDGMEKSVLAAKDR
LGYVHIGESHRGYLGTGNVDFDTFFAALKKIGYQGPITFESFSSEVVDPN
LSNTLCVWRNLWHDSDDLAKKALQFMKERYSGNPQRSP.

In some embodiments, the polypeptide comprises or consists of the amino acid sequence:

(SEQ ID NO: 15)
MKFGIHSMVWVGDWSPEHARHAIASSAEAGYDLIELAAIDPATFDVDLTA
KLLAEHGIQASASLGLDAATDVSSEDPDVVAAGRARLRDALALVRDTGGT
MLCGVIYSALRKYDAPVTERGLANSQETIAALADEAAASGITIGLEFCNR
YETNVLNTTAQTLAFIAAVDRPNVMAHLDTYHMNIEEPSMSEPVLAAAGR
LGYVHVGESHRGAPGTGSIPWDEFFGALRECGYDGIVTFESFSSEVVHPS
FSNTLAIWRNLWTDNRKLATGALAFMRAHLRAAGGRGA.

In some embodiments, the polypeptide comprises or consists of the amino acid sequence:

(SEQ ID NO: 16)
MKLGIHALVWVGGWTPEEAELAISSTARAGYDLIEIPALDPSRIDVASTR
EMLERHGLQASVSLGLAPDTDINSEDVACVDRGRDVLMRALEVSSGIGST
YLGGVIFGAMTKYSAPTTERARANSVAVIKELAQEARGCGTTIGLEFVNR
YESNLLNTAQQTLDYLDLVGEDNVVVHADVYHMNIEETDFRTPILACGDR
LGYVHVGESHRGYLGTGSVNFPEFFGALKEVGYAGPITFESFSSSVVDPL
LSNTLAIWRNLWSDSTDLATQAHAFISAGLADDGGRGA.

In some embodiments, the polypeptide further comprises a tag. In some embodiments, the tag is an N′-terminal tag. In some embodiments, the tag is a C′-terminal tag. In some embodiments, the tag is an affinity tag. In some embodiments, the tag is a poly Histidine tag. In some embodiments, a poly Histidine tag comprises 4 to 10 Histidine residue. In some embodiments, the poly Histidine tag comprises 6-8 Histidine residues. In some embodiments, the poly Histidine tag comprises 6 Histidine residues.

The terms “homology” or “identity”, as used interchangeably herein, refer to sequence identity between two amino acid sequences or two nucleic acid sequences, with identity being a stricter comparison. The phrases “percent identity or homology” and “% identity or homology” refer to the percentage of sequence identity found in a comparison of two or more amino acid sequences or nucleic acid sequences. Two or more sequences can be anywhere from 0-100% identical, or any value there between. Identity can be determined by comparing a position in each sequence that can be aligned for purposes of comparison to a reference sequence. When a position in the compared sequence is occupied by the same nucleotide base or amino acid, then the molecules are identical at that position. A degree of identity of amino acid sequences is a function of the number of identical amino acids at positions shared by the amino acid sequences. A degree of identity between nucleic acid sequences is a function of the number of identical or matching nucleotides at positions shared by the nucleic acid sequences. A degree of homology of amino acid sequences is a function of the number of amino acids at positions shared by the polypeptide sequences.

The following is a non-limiting example for calculating homology or sequence identity between two sequences (the terms are used interchangeably herein). The sequences are aligned for optimal comparison purposes (e.g., gaps can be introduced in one or both of a first and a second amino acid or nucleic acid sequence for optimal alignment and non-homologous sequences can be disregarded for comparison purposes). The optimal alignment is determined as the best score using the GAP program in the GCG software package with a Blossum 62 scoring matrix with a gap penalty of 12, a gap extend penalty of 4, and a frame shift gap penalty of 5. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences.

In some embodiments, % homology or identity as described herein are calculated or determined using the basic local alignment search tool (BLAST). In some embodiments, % homology or identity as described herein are calculated or determined using Blossum 62 scoring matrix.

In some embodiments, the polypeptide comprises or is characterized by 3-epimerase activity. In some embodiments, the polypeptide comprises or is characterized by D-psicose 3-epimerase activity.

As used herein, the term “3-epimerase” refers to enzymes that catalyze the isomerization of C3 position of ketose monosaccharides and includes the D-Tagatose-3-epimerases (DTEases) family of enzymes. Normally, D-allulose is produced through the isomerization of D-fructose under the catalysis of DTEase family enzymes. DTEase family enzymes include DTEases, D-psicose 3-epimerases (DAEase), and ketose 3-epimerase. All of these enzymes have the same characteristics that catalyze the conversion of D-fructose to D-allulose and possess a highly conserved activity center and key amino acid residues with similar features.

As used herein, the term “D-psicose 3-epimerase” refers to an enzyme having D-psicose 3-epimerase activity (EC 5.1.3.30) that promotes the conversion of D-fructose to D-psicose by catalyzing the epimerization at the C3 position of D-fructose. The enzyme is highly specific for D-fructose and shows very low activity with D-tagatose (cf. EC 5.1.3.31, D-tagatose 3-epimerase). The enzyme requires and/or is enhanced in the presence of a metal ion cofactor (e.g., Mn²⁺, Co²⁺, Mg²⁺). D-psicose 3-epimearse is also known as DPEase, DPE, D-allulose 3-epimerase, or DAE.

According to some embodiments, there is provided a polynucleotide encoding the polypeptide of the invention. In some embodiments, the polynucleotide comprises a nucleic acid sequence comprising a sequence selected from SEQ ID Nos.: 17-32.

In some embodiments, the polynucleotide is an isolated polynucleotide. In some embodiments, the polynucleotide is a DNA molecule. In some embodiments, the polynucleotide is an isolated DNA molecule. In some embodiments, the DNA molecule is an isolated DNA molecule. In some embodiments, the DNA molecule is a complementary DNA (cDNA) molecule.

As used herein, the terms “isolated polynucleotide” and “isolated DNA molecule” refers to a nucleic acid molecule that is essentially free from contaminating cellular components, such as carbohydrate, lipid, or other proteinaceous impurities associated with the nucleic acid in nature. Typically, a preparation of isolated DNA or RNA contains the nucleic acid in a highly purified form, e.g., at least about 80% pure, at least about 90% pure, at least about 95% pure, greater than 95% pure, or greater than 99% pure. In some embodiments, the isolated polynucleotide is any one of DNA, RNA, and cDNA. In some embodiments, the isolated polynucleotide is a synthesized polynucleotide. Synthesis of polynucleotides is well known in the art.

The term “nucleic acid” is well known in the art. A “nucleic acid” as used herein will generally refer to any molecule (e.g., a strand) of DNA, RNA or a derivative or analog thereof, comprising nucleotides. Nucleotides are comprised of nucleosides and phosphate groups. The nitrogenous bases of nucleosides include, for example, naturally occurring purine or pyrimidine nucleosides as found in DNA (e.g., an adenine “A,” a guanine “G,” a thymine “T” or a cytosine “C”) or RNA (e.g., an A, a G, an uracil “U” or a C).

The term “nucleic acid molecule” includes but is not limited to single-stranded RNA (ssRNA), double-stranded RNA (dsRNA), single-stranded DNA (ssDNA), double-stranded DNA (dsDNA), small RNAs, circular nucleic acids, fragments of genomic DNA or RNA, degraded nucleic acids, amplification products, modified nucleic acids, plasmid or organellar nucleic acids, and artificial nucleic acids such as oligonucleotides.

In some embodiments, the polynucleotide comprises or consists of the nucleic acid sequence:

(SEQ ID NO: 17)
ATGAAGTTTGCGATTTGTAATGAGACTTTCCAGCCAGACGCTCCGCCGTT
ATGGCCTCTTGAGAAAGCATTCGCTTATGCGCGTGAACTTGGTTACACCG
GTATCGAATTCGCACCTTTTGATGCGACAGACGTAAGTGCAGAACGGCGG
GCCGAGGTGCGCGAGCTTCTTGAAAAATACCAGCTCGAATCTATTGGTTT
ACATTGGCTGTTAGCTAAAACAGAGCTCACTTCCCCGGATGCAGCTGTAC
GGGATCGGACTTCCGAGTATCTGGCGGAACTGGCACGTCTTTGCCGGGAT
CTTGGCGGTTCCGTGATGTGCGGCGTCCTGGGTTCGCCACAACAGCGGAA
CTTACTGCCGGGGGTAACTCACGACCAGGCAATGGAATATGCAGCCGATG
TAATCCGCGCGGCGATGCCGGCCTTAGAAGACCACGGGGTAACTCTCGCT
CTCGAACCGTTAGGCCCGGCAGAGGGGGACTTCTTGCTGACTGCCGAATC
GGGTATTCAGTTAGCTCAGATGATCGACTCCCCGCATGTACGTCTTCATT
TGGATGTTAAAGCAATGAGTCTCGAAGCGAAGCCGATTCCAGATATTATC
CGGGATTCTGCAGATTACTTGGCGCACTTCCATGCAAACGACCCAAATAA
GCGGGGCCCTGGCATGGGCGACGTTGACTTCGTCCCTATCTTTCAGGCTT
TACAGGAAATTAACTACCAAGGCTGGGTATCCGTGGAAGTATTTGATTAT
GAACCAGGTGTGGTTGAACGCCTTGCCCGTGAATCCATTTCATACATGCA
ACGGTGTCTGGACAAACTTTATTCCTCGGTATCGGGTGGCGAACGCCCGC
CTCCAGACAAC.

In some embodiments, the polynucleotide comprises or consists of the nucleic acid sequence:

(SEQ ID NO: 18)
ATGAAATTTGCGATTTGCCAGGAACTGTTCGAGCCGGATGCGCCGCCGCT
CACAGACTGGGCCGAGCAATGTCGGATCATCGCGGAGGTCGGCTACACCG
GGATTGAGGTTGCTCCTTTTGATTTAGCTTCAGTACCGGCGGCAACACTT
ACTGAAATGCGGAATACGGCAGAGCGCCATGGCCTTGAAATTATCGGGCT
GCATTGGTTGCTCGCGAAGACGAATCTTACATCTCCTGATGCCGCCGTGC
GGACCGCTACAGCCGACTACTTGAAGTTATTAGCGGATACCTGCGCCGCT
CTGGGGGGTCGGGTGCTCGCCGGGGTTTTTGGCTCACCTCAACAACGGAA
CTTGTTGGCAGGCGTAACAACCGAACAAGCAATGGATTTTGCTGCAGAAG
TATTTCGGGCTGCCATGCCAACGTTTGCGGAGCGGGATGTGGTCCTCTGC
ATGGAGCCGCTCACACCGAAGGAGACTGATTTTATTAACACCTGCGCTGA
CGCTGTTGCGCTGATGCAAATGGTTGACCACCCAAGTTTCGTCCTTCACC
AAGATGTAAAAGCAATGCTGCTTGCCGAGAGTGATGTGCCAGAGCTGATC
CACCGTCATAAAGATATCTGCGGCCATTTTCACGTTAATGACACTAATTT
ACTTGGGCCAGGTATGGGGGAAACGGACTATCATCCAATTCTGAAGGCGT
TACTGGAAGTCGGTTATGACGGTTGGGTTTCCGTCGAAGTGTTCGACTAT
TCACCAGGGGCAGTCGAGAAGATTGCGAAGAAATCGATGGATTATATGCA
GCAAGTATTGTCCGATCTCTATAAGGCGGTTTCAGGTGTGTCTGGTCCGC
CATGCGATAAC.

In some embodiments, the polynucleotide comprises or consists of the nucleic acid sequence:

(SEQ ID NO: 19)
ATGAAGTTAGGGATTCACAGTTTCGTATGGACTGGGGGGTCCACTCAAGA
AGACTTAGAAGGTGCTATGGAAAAGAGCCATGAATTAGGCTATGATTTAA
TCGAGTTTCCGCGTCTGGACCCGAAGAAATTCGATGTAGCTTGGCTCGCC
AAACGCCTGGAAGATTCTGGTCTGAAAGTAGTGGTGACCATGGGTTTACC
ACTTGATGGCGATATCTCATCAGAGGACGCAGACGTGGTGAAATCTGGTG
AACAAATTCTGGATGACGCTGTAGCGGTCACCCGTGATTTAGGTGGCGAG
AAACTTGGTGGTATCTTGTTTTCGGCCCACGGGAAGTATCAATCAATGCC
AACACGGAAAGGTTGGGATAATAGTGTTGCCGTAATGCGGAAAGTAGCAG
ATCGTGCGAAGGCGGCGGGTGTAACACTCAACCTGGAGATTGTCAATCGG
TTCGAGTCAAACTTGTTGAATACGACAGCACAGGGTTTAGCATTTATCGA
AGACACGGGTTCTGATAACATTTTTTTACATTTAGATACGTTTCACATGA
ACATTGAGGAAGCTGATCCGGCACAAGCCATCCGTCATGCCGGCGACAAA
CTTGGTTATTTCCATATTGGGGAGTCGAACCGGGGGTATTTAGGCGCGGG
GACTATCGACTTCCCGCCGATTTTCGACGCGCTGGTAGCGATTGGCTACG
AAGACTACGTCACCTTCGAGTCATTCTCCTCCGAAGTAGTCGACAAAGAT
TTATCTATCACAACGGGTATTTGGCGGAATACCTGGACAGATAATGTCGA
GGTGGCACGTCACGCTAAAGCATTCATTGAGGCACGGTATGGTGAAGCTC
GTCGTAAGGCACAGATCGCG.

In some embodiments, the polynucleotide comprises or consists of the nucleic acid sequence:

(SEQ ID NO: 20)
ATGCGGTTGGGTATTCACTCGTTCGTGTGGACGGGTGGCCAGACGCAGGA
GGGGTTAGAAATGGCGCTGGAGAAGTCAGCGGAACACGGCTACCGGTTAA
TCGAGTTTGCGTACCTGCGGCCGGAAAAGTTCGACCTGGATCGTCTCGCA
AAGAAAGCCCAATCCCTCGACTTAGAGATTGCAGTAACAATGGGTTTACC
TCTCGATGCGGATGTATCCTCTGAAGATGCAGATGTAGTCGCTCGGGGGA
AAGCTATGTTAGCGGACGCGGTCAAGGCTGTACGTGATATTGGGGGCAAT
AAATTAGGGGGTATTCTCTATTCAGCCCACACCAAATATAATCGTATGCC
GACGCAGCGGGGCTGGAAGAATAGTGTAGAAGCTATTGCAAAGACTGCGG
AGATTGCCAAGGCGTGCGGCGTTGACTTAGTGCTGGAAGTGGTCAATCGG
TTTGAGACAAATTTGTTGAACACAACCGCGCAAGGTCTTAAGTTCATTGA
AGACGTAGGCTCCGACGATGTACGTTTACATCTCGATACCTTCCACATGA
ATATTGAAGAGGCAGATCCGGCAGCGGCTATTCGTCTTGCAGGGGATAAG
CTGGGCTACTTTCACATTGGTGAAAACAACCGGGGGTACCTGGGTGACGG
GGTTATCGACTTCGATGCTATCTTCGACGCGCTTTTGGACATCGGTTATG
ATCGGGACATCGTCTTTGAGTCTTTCTCCAGTGCCGTAGTTGACGAGGGG
CTGTCGCTGGCATGTGCAATTTGGCGTGATACGTGGACTGATAATGATGA
ATTGGCGGCACATGCGAAGCAATTCATTGAGTTAAAATACGATGAAGCGC
AACGTCGTCGTGCTACTAAT.

In some embodiments, the polynucleotide comprises or consists of the nucleic acid sequence:

(SEQ ID NO: 21)
ATGCGCTTAGGGATTCACTCTTTTGTCTGGACTGGGGGGCAAACGCAAAG
TATGTTAGAAGAGGCAATGGAAAAATCAGCAAGCTGCGGGTATAATCTGA
TTGAGTTCGCTTACCTCCGCCCTGAGAAGTTTGACCTCGATGCGTTAGCA
AAGCGTGCCCAGGCATTAAAATTAGACATTGCGGTCACCATGGGGTTACC
TTTCTCTCACGATGTCTCATCTGAGGACGCTGACACTGTTAAGGCAGGTG
AGGGGATGCTCGCAGATGCAGTGCGCGCCGTTCGGGATATCGGCGGTTCA
AAGCTCGGTGGTATTTTGTACTCCGCTCACTCCAAGTACAGCCGGATGCC
GACGAAGAAAGGGTGGGATAATTCCGTAGCGGCCATCGCCAAGACGGCGG
ACTTAGCTAAGTCAGCCGGTGTAGATCTTGTTCTTGAGGTTGTTAACCGT
TTTGAGACTAATCTGCTTAATACGACAGCACAGGGCTTGGCCTTCATTGA
GCAGGTTGGGTCTGAACACGTGCGTCTCCATCTTGATACCTTCCACATGA
ATATTGAAGAGGCGAATGTCGCAGCCGCGATCCGTTTAGCTGGTGATAAG
ATCGGGTACTTCCACATTGGTGAAAGTAATCGGGGTTATCTCGGGGATGG
TAATATTAACTTTGACCTTATCTTTGATGCTCTGCTCGATATTGACTATC
AACGTGACATCACATTTGAGTCCTTTTCTGGCGCGGTTGTTGATGAGGGG
CTGTCTCTGGCTTGCGCGATTTGGCGGGACACCTGGACAGAAAATATGCC
TTTAGCCCAGCACGCAAAACAGTTCATCGATCTTAAGATGGACGAGGCCA
AGCGCCGTCGTGCAACGGTG.

In some embodiments, the polynucleotide comprises or consists of the nucleic acid sequence:

(SEQ ID NO: 22)
ATGCGCCTTGGTATTCACTCTTTTGTCTGGACGGGTGGCCAGACGCAAGA
AGGGTTAGAGATGGCGTTGGAGAAGAGCGCTGAACATGGTTATCGGTGTA
TTGAGTTTGCGTACCTCCGGCCAGAGTTATTCGATTTGGATCGGTTGGCG
CGTAAATCGCAGAGTCTCGACGTGCAAATTGGGGTCACAATGGGTTTGCC
ACTTGACAAAGACGTAAGCTCCGAAGACCCTGATGTAGTTGCGGCGGGGA
AGCAGATGCTCGCCGACGCGGTACGCGCAGTCCGGGACGTAGGCGGTGAC
AAGCTGGGCGGCATCTTATATAGTGCGCACACAAAATATAACCGCCAACC
TACAGCGGATGGTTGGAAAAATTCGGTTGAAGCCATCGCCGAGACTGGGG
AAATCGCTAAGGCCGCCGGTGTAGATTTGGTGTTGGAAGTGGTGAACCGC
TTTGAAACCAATCTGCTCAATACAACCGCGCAGGGGTTAAAGTTCATTGA
TGATACAGGCAGCGATCATGTCCGTCTGCACCTTGATACATTTCACATGC
AAATTGAGGAAGCTAATCCTGCTGCGGCCATTCGTCTGGCTGGCGATAAG
CTGGGGTATTTTCACATCGGTGAGTCCAACCGGGGCTACTTGGGGGACGG
CACTATCGACTGGGACCCTATCTTTGACGCTTTGCTGGATATTGGTTATG
ATCGTGATGTGGTTTTCGAATCGTTCAGCACCGCTATCGTCGATGAAGGT
CTTAGTTTGGCCTGCGCAATTTGGCGCGATACCTGGACGGAAAACGATGA
GTTGGCAGCACACGCTAAGCAATTCATCGAACTCAAGTGGGCCGAGGCTC
AGCGTCGGCGCGCGACCAAC.

In some embodiments, the polynucleotide comprises or consists of the nucleic acid sequence:

(SEQ ID NO: 23)
ATGCGCTTAGGCATTCATTCTTTCGTCTGGACCGGGGGTCAAACCCAGGA
GGGGTTGGAAATGGCTTTGGAAAAATCTGCAGAGCACGGCTATCGGACTA
TCGAGTTTGCGTACCTGCGCCCAGAATTGTTTGACTTGGACCGGCTGGCT
CGTAAAGCGCAGTCCCTGGACGTGGAGATCGGGGTAACGATGGGCTTACC
GTTAGATAAGGATGTGTCTTCGGAGGACCGTGACGTTGTAGCACGGGGTA
AGCAAATGCTCGCCGATGCAGTCCGGGCAGTTCGTGACATTGGGGGCAAT
AAATTGGGGGGTATCTTATACTCCGCACATACTAAGTACAATCGGCTGCC
GACTGCAGATGGGTGGAAAAACTCGGTGGAAGCGATCGCGGAAACTGGGG
AGATCGCCAAAGCGGCTGGCGTTGACCTTGTGCTTGAGGTGGTCAACCGC
TTCGAGACGAACCTCCTGAATACCACCGCACAGGGTCTTAAATTCATCGA
CGACGTGGGTAGCGATCACGTTCGCCTTCATTTGGACACGTTCCACATGA
ACATCGAGGAAGCGAATCCTGCCGCTGCGATTCGCCTTGCTGGTGATAAA
CTCGGTTATTTCCATATTGGCGAGTCTAACCGCGGGTACCTCGGGGATGG
GAATATCGACTTCGACTTAATTTTTGATGCTTTGCTGGATATTGACTACC
AACGTGACGTTGTCTTTGAATCATTTAGTACTGCAATCGTTGATGAGGGG
CTTAGTTTAGCATGCGCTATCTGGCGGGATACGTGGACTGAAAACGATCC
TCTGGCGGCGCACGCGAAACAATTCATTGAACTTAAGTACGCTGAGGCAC
AGCGCCGTCGCGCCACCAAC.

In some embodiments, the polynucleotide comprises or consists of the nucleic acid sequence:

(SEQ ID NO: 24)
ATGCGCTTAGGCATTCATTCATTTGTATGGACGGGCGGCCAAACGCAGGA
AGGCTTAGAAATGGCGCTCAACAAGACTGCTGAGCACGGCTATCGCACAA
TTGAGTTCGCTTACCTGCGCCCAGAGAAATTTAACTTAGATCGGCTTGCC
AAGCTTGCGCAGAGCCTTGATGTCGAAATCGGTGTGACTATGGGGCTGCC
GCTTGATAAGGATGTGAGCAGTGAGGATGCAGCAGCCGTTGCAGCTGGGA
AACAAATGCTTGCAGATGCGGTTCGCGCTGTTCGTGATATTGGGGGCAAT
AAATTGGGGGGCATTTTATACTCCGCCCACACCAAGTATAACCGCCAGCC
GACTAAAAAAGGCTGGGACAATAGTGTGGCCGCAATCGCAGCCACAGCGG
AGATTGCCAAAGAGGCGGGCGTCGATTTGGTCCTTGAGGTGGTGAATCGC
TTTGAGACAAACTTATTAAACACTACTGCGCAGGGCCTGAAGTTTATTAG
TGAAGTCGGGTCGGACCATGTACGGCTTCATCTGGATACATTTCACATGA
ACATCGAGGAGGCCAACCCAGCAGCCGCGATTCGGTTAGCGGGGGATAAA
ATTGGGTACTTCCACATCGGCGAGAGCAATCGCGGTTACCTTGGCGATGG
GGTTATTAACTTCGATCTCATTTTTGACGCATTGACCGATATCGACTACA
AGCGTGATATCGTCTTCGAAAGTTTTTCGACCGCCGTGGTAGATGAAGGG
CTCTCTCTCGCATGTGCGATTTGGCGTGACACCTGGACAGATAACGATGA
GCTCGCCGCTCATGCGAAGCGGTATATTGAGCTCAAGTACGATGAAGCCA
AACGTCGCCGCGCCACAAAC.

In some embodiments, the polynucleotide comprises or consists of the nucleic acid sequence:

(SEQ ID NO: 25)
ATGAAGCTTGGCATTCACTCATTCGTGTGGACCGGGGGTACCACCCAGGA
AGATCTTGAAGGCGCGATGGAGAAGAGCCATGATCTTGGTTACGGCTTGA
TTGAATTTCCACGTCTCGATCCTAAAAAGTTTGACGTGTCCTGGCTCGCT
AAACGCTTGGAAGATTACGGCCTGAAAGTGGTAGTCACTATGGGCCTCCC
TTTAGATGGGGACATCAGTTCTGAGGACGCCGCGGTTGTCAAACGGGGTG
AGCAGATCCTTGACGACGCCGTTGCCATCACTCGTGATCTCGGCGGCGAG
AAATTGGGCGGCATTATCTTCTCCGCTCACACGAAGTACCAATCAATGCC
TACTCGCAAAGGGTGGGACAACAGTGTGGCTGCTCTGAGCCGCGTGGCTG
ATCGCGCTAAAGCAGCAGGGGTTAGCCTGAATCTTGAAATCGTGAACCGT
TTTGAATCAAACCTTCTCAACACTACTGCCCAAGGGCTCGCATTCATTGA
AGAAACTGGCTCTGATAATATCTTTCTCCACTTGGACACCTTTCACATGA
ACATCGAGGAAGCTGACCCAGCCCAAGCTATTCGCTTAGCCGGTGACAAG
CTGGGTTACTTCCACATTGGGGAATCAAATCGGGGCTACTTAGGGTCAGG
TGTGATCAACTTCCCGGCAATTTTCGACGCCCTTGTCGCAATTGGCTACG
AGGATTGGATTACGTTCGAGAGCTTTAGCAGTGAGGTGGTAGATGAAGAT
CTTAGTATTACCTGTGCTATCTGGCGCAATACTTGGACTGATAACGTAGA
GGTAGCCCGGCTCGCCAAAGCGTTTATCGAAGCGCGTTACGGGGAAGCCC
GTCGTAAGGCTCAGACCGCG.

In some embodiments, the polynucleotide comprises or consists of the nucleic acid sequence:

(SEQ ID NO: 26)
ATGCAAATCGGTGCGCATGGGTTAGTCTTCACCGGTACCTTCGATGAGGC
GGGGCTCCGCCGCGCGATCGAAGGCACTAAACACGCTGGTTTCGACCTTA
TTGAGATCCCGCTCATGGACGTAGATGGCTTCGACTCTGCTTTAGCTGGT
CGGATGTTGGCCGACAACGATCTCGCGGTCACAGCGTCTCTGGGCTTGAC
TGCGGCAACAGATTTAACGAGCGAAGATCCTGCTGTTGTAGCTGCTGGGG
AACGGATGTTAGAGCGCTGCTTAGACCACGTCGCTGCTATGGGCGGGGAC
GTACTCTGTGGCGTGATTTATAGCGCTATGCAAAAATATATGGCACCTGC
GACCGAACGGGGGATCGCTAATAGTCAAGCTGCTATTGCACGGCTTGCGG
AGAAGGCGGCGGAGCGCGGTATTCGTCTTAGTCTCGAAGTTGTTAACCGG
TACGAATCAAACGTCTTTAACACTGGGCGTGGTGCGCTTGCCTTCCTCGA
CGGTGTTGGTCGGGATGACGTCTCTGTCCATTTAGACACCTATCACATGA
ATATTGAAGAGAGTGATCTCTTTCAACCGGTCCATGATGTTGCAGGTCGC
TTGGGGTACGTTCATATTGGTGAATCGCATCGCGGCTACTTAGGCACCGG
TACTGTGGACTTCGGTACTTTCTTTCGTGCCCTGCATGACGTGCGTTATG
ACGGGCCAGTCGTCTTTGAGTCCTTTTCGTCCGCAGTGGTCAGTGCAGAA
CTCTCAAATACATTGGGGATTTGGCGCAACCTCTGGGAAGACAGCGACGA
TTTGGCAGCACATGCTAATCGTTTCATTCGTGACGGCCTTCACGCTGTCC
GTACAATTGATTTG.

In some embodiments, the polynucleotide comprises or consists of the nucleic acid sequence:

(SEQ ID NO: 27)
ATGGACATTGGGTGTCATGGTTTAGTGTGGACCGGCTCATTTGACGCTCA
GGGCATCCGTCTCGCGGCCCAGAAAACCAAGGAAGCCGGTTTCGATCTGA
TTGAATATCCACTTATGGATCCGTTCACCTTTGACACTGAGGCCGCGGTG
AGCGCCCTTGCGGAGCATGGCTTGAAAGCAAGTGCGAGCCTCGGTCTGTC
GGACTCAACAGATGTGACTTCTACCGATCCGGAAGTGGTCGCGGCAGGCG
AAGCCTTACTGCTGCGGGCGGTCGATGTTCTTGCTGATATGGGTGGTACA
CACTTGTGTGGCGTAATCTACAGTGCTATGAAAAAGTATATGGAGCCTGT
AACAGCCGAAGGCTTGGAGAACAGCAAGCGGACTATCGGCCGTGTCGCTG
ATCATGCAGCGGACCGTGGTGTAAACGTGAGTCTTGAGGTGGTTAATCGG
TATGAGACCAATGTATTGAACACTGCGCGGCAGGCGATCCGGTTCGTTGC
GGACGTAGACCGGCCGAATCTCGGCATTCATTTGGACACGTACCACATGA
ACATCGAGGAGAGTGACATGTTTGCCCCTGTCTTAGACGCGGGTGAAGTC
CTCCATTACGTTCATATCGGTGAATCGCATCGCGGCTACCTTGGCACAGG
GTCCGTAGATTTCGACAATTTTTTCAAAGCCCTCGGCCGTATTGGTTACG
ATGGTCCAATCACATTCGAGTCGTTTTCGAGCGCCGTAGTTGCTCCAGAC
CTTTCGCGTATGTTAGGCATCTGGCGTAACTTATGGACCGACAATGCAGA
TTTAGGCGCACATGCGAATCGGTTCATCCGTGATAAGCTTACCGCAGTTG
AAACTATTAGCCTC.

In some embodiments, the polynucleotide comprises or consists of the nucleic acid sequence:

(SEQ ID NO: 28)
ATGGAGTATGGGGGCCACGCGCTTGTATGGGCAGGCGACTGGTCTCCAGA
GGGCGCACGGCAAGCTATTTCGGGTGCGGCCCGGGCAGGTTATGACTACA
TTGAGATCGCCCTCCTCGACCCTTGGAAAGTTGATGTAGCGATGACCAAA
GATCTGCTCCAGGAATACGGTCTTCGTGCTCATGCGAGCTTAGGTCTTAG
TCCGACCACCGATGTAACGTCAACCGACCCAGCTATTGTAGCGAAGGGGG
ATGAACTGCTGCGTAAGGCTACAGATGTTCTCCATGCACTCGGGGGGACG
GAACTTTGCGGCGTTATTTACTGTGCGTTAGGTAAGTACCCAGGGCCAGC
CTCAAAAGAAAACCGCGCAAATAGCGTAGCAGCAATGCAACGCTTGGCTG
ACTATGCGGCTGACAAGGGCATTAATATTAATTTAGAAGTAGTCAACCGG
TATGAGACAAACATTATGAATACAGGGATCGAAGGCTTAAAATTTCTGGA
GGAGGTGAACCGGCCAAACGCGTACCTCCACTTGGACACTTATCACATGA
ACATTGAAGAGGATGGTATGGAGAAGGCTGTTTTAGCCGCAGGGGATCGT
CTCGGGTACGTGCATATCGGGGAGTCCCACCGTGGCTATTTGGGTTCTGG
CAACGTGGACTTTGATACGTTCTTCGCTGCATTAAAGAAGATTAACTATC
AGGGCCCGATTACTTTTGAGTCTTTCTCTTCTGCCGTAGTCGACCCTGCG
CTGTCAAACGCGCTTTGCGTCTGGCGCAATTTGTGGAGCGATTCCGACGA
CCTTGCGAAGAAAGCATTGAAGTTTATGAAGGCTCGTTATTCGGGGAACT
CTCAACGCAGCCCG.

In some embodiments, the polynucleotide comprises or consists of the nucleic acid sequence:

(SEQ ID NO: 29)
ATGGAATACGGCGGTCATGCCTTAGTGTGGTCTGGGGACTGGACGCCGGA
AGGTGCTCGTAAGGCGATTTCCGGTGCAGCACGGGCGGGTTACGACTATA
TTGAAATCGCACTCCTTGATCCTTGGAAAGTAGATGTCGCCTTAACTAAA
GATTTATTGCAAGAGTACAATCTCCGTGCTCACGCATCGTTAGGTCTCTC
TGCAGCAACAGACGTCACTAGTACGGACCCTGCCATTGTAGCCAAAGGCG
ACGAGCTGTTACGCAAGGCGACGGACGTTCTTCACGCCATCGGGGGCACA
GAACTCTGCGGTGTAATCTACTGTGCACTGGGCAAGTATCCAGGGCCAGC
CAGTAAAGAAAATCGCGCGAATAGTGTCGCGGCTATGCAACGGCTGGCGG
ACTACGCAGCGGACAAGGGGATTAACATCAACCTGGAGGTTGTCAATCGC
TATGAGACAAACATCATGAACACTGGCCTGGAAGGGCTGGCATTTCTGGA
CGAGGTCAATCGTCCTAATGCGTATCTCCACTTAGATACTTATCACATGA
ACATCGAAGAAGATGGCATGGAAAAGTCGGTACTCGCCGCCAAAGATCGG
CTGGGTTACGTGCACATTGGCGAGTCTCACCGCGGCTATCTCGGGACTGG
CAATGTCGATTTCGATTCCTTCTTTGCTGCATTAAAAAAGATTGACTATC
AGGGTCCAATCACTTTTGAGAGCTTCTCCTCGGAGGTAGTTGACCCTAAG
CTGAGTAATACGTTGTGTGTATGGCGGAACCTCTGGCATGACTCTGACGA
TCTCGCGAAAAAAGCACTCCGGTTCATGAAGGAGCGGTATCCAGGGAACC
CACAACGGTCGCCT.

In some embodiments, the polynucleotide comprises or consists of the nucleic acid sequence:

(SEQ ID NO: 30)
ATGAAGTACGGTGGCCATGCCTTGGTATGGAGCGGTGATTGGACGCCAGA
AGGCGCTCGCCAAGCCATCTCGGGGGGGGCTCGCGCTGGGTACGACTATA
TCGAAATCGCGTTACTGGACCCATGGAAAGTAGACGTAGCTTTGACCAAG
GACCTCCTGCAGGAGTTTAACTTACGTGCTCACGCGAGTCTGGGCCTGAG
CGCAGCCACGGATGTCACCAGTACAGACATGGCTATCGTGGCCAAGGGGG
ATGAGCTGCTTCGCAAAGCCACCGACGTACTGGCGGCGATCGGCGGGAAA
GAGCTGTGTGGGGTTATTTACTGTGCCCTTGGCAAATACCCAGGTCCTGC
CTCGAAAGAGAATCGGAACAACAGTGTCGCTGCGATGCAACGTCTTGCGG
ATTATGCCGCAGATAAGGACATCAATATCAATTTGGAGGTAGTTAACCGC
TACGAGACAAACATTATCAACACAGGTATCGAAGGTTTAGCATTTCTGGA
TGAGGTGGATCGCCCGAATGCCTATCTTCACTTGGACACTTACCACATGA
ATATCGAGGAAGATGGGATGGAGAAGTCCGTACTCGCGGCTAAAGACCGC
CTCGGCTATGTTCACATCGGCGAATCCCATCGGGGCTATCTTGGCACAGG
CAATGTAGATTTTGATACTTTCTTTGCAGCCTTAAAGAAAATTGGGTATC
AAGGGCCAATTACATTTGAGTCCTTCAGTTCTGAGGTCGTTGACCCTAAC
CTGTCGAATACGCTGTGTGTCTGGCGCAATTTGTGGCATGACTCGGATGA
CCTCGCGAAAAAGGCCTTGCAATTCATGAAGGAGCGCTACTCGGGTAACC
CGCAACGGAGTCCG.

In some embodiments, the polynucleotide comprises or consists of the nucleic acid sequence:

(SEQ ID NO: 31)
ATGAAGTTTGGGATCCATTCAATGGTGTGGGTGGGGGACTGGAGTCCTGA
ACACGCGCGCCATGCCATCGCCTCAAGTGCTGAAGCGGGCTACGATTTAA
TCGAGTTGGCTGCAATCGACCCGGCTACGTTCGATGTAGATCTGACCGCG
AAACTTCTTGCTGAGCACGGGATTCAAGCGTCGGCTTCCCTTGGTCTTGA
TGCGGCGACCGACGTATCAAGCGAAGACCCGGATGTGGTGGCCGCTGGGC
GCGCTCGCTTGCGGGACGCTTTGGCTCTCGTCCGGGATACCGGTGGCACG
ATGCTTTGTGGCGTTATTTACTCCGCTCTGCGGAAGTACGACGCACCGGT
AACTGAGCGTGGTTTAGCCAATTCACAAGAGACCATTGCAGCACTCGCGG
ACGAGGCCGCGGCTTCCGGTATTACTATCGGGCTGGAGTTCTGCAACCGT
TATGAAACGAACGTACTTAACACGACCGCCCAGACTTTGGCGTTCATCGC
AGCCGTGGATCGCCCTAACGTTATGGCTCACTTAGATACCTACCACATGA
ACATTGAAGAACCGTCTATGTCTGAGCCTGTTCTTGCAGCGGCTGGCCGC
CTTGGTTACGTCCATGTTGGTGAGTCTCACCGCGGCGCGCCTGGTACTGG
TAGTATCCCATGGGACGAGTTCTTCGGTGCCCTCCGTGAGTGTGGGTATG
ATGGTATTGTGACTTTTGAGAGTTTTTCTTCTGAAGTTGTTCACCCTAGT
TTTTCCAACACATTGGCCATCTGGCGGAACCTGTGGACTGACAATCGCAA
ACTTGCAACCGGTGCTCTTGCTTTTATGCGGGCCCATTTGCGGGCCGCTG
GCGGTCGCGGGGCC.

In some embodiments, the polynucleotide comprises or consists of the nucleic acid sequence:

(SEQ ID NO: 32)
ATGAAACTCGGGATTCATGCATTAGTGTGGGTAGGTGGTTGGACACCTGA
GGAAGCTGAACTCGCCATTAGTAGCACGGCGCGCGCAGGGTACGATCTCA
TTGAAATCCCTGCGCTGGACCCGTCGCGTATTGACGTTGCTTCCACCCGG
GAGATGCTTGAGCGCCATGGTTTGCAAGCAAGCGTGTCCCTGGGTCTCGC
TCCTGACACCGACATCAATTCGGAAGATGTAGCGTGTGTAGATCGTGGCC
GGGATGTCTTGATGCGTGCACTGGAGGTTAGTTCTGGCATCGGTTCCACA
TACCTTGGGGGCGTAATTTTCGGGGCTATGACTAAGTACAGCGCCCCTAC
CACAGAGCGGGCCCGCGCAAATAGCGTGGCGGTAATCAAGGAGTTGGCCC
AAGAAGCCCGTGGCTGCGGGACAACGATTGGGCTTGAATTTGTTAATCGG
TACGAGAGTAACTTATTAAATACTGCTCAACAAACCCTGGACTACTTGGA
TCTCGTAGGGGAAGATAACGTGGTGGTACACGCTGATGTATATCACATGA
ACATCGAGGAGACGGATTTCCGCACACCGATTCTGGCTTGCGGCGACCGT
CTGGGCTATGTTCACGTAGGCGAATCCCACCGTGGTTACCTGGGTACAGG
TTCGGTAAACTTTCCTGAATTTTTCGGTGCTTTGAAGGAAGTCGGGTATG
CAGGGCCTATCACCTTTGAATCATTCTCCTCCAGCGTCGTTGACCCTCTG
TTGAGCAATACATTGGCGATCTGGCGTAATCTTTGGAGTGACAGCACAGA
TCTGGCCACTCAGGCGCATGCATTTATCTCGGCAGGCTTGGCGGATGATG
GGGGGCGTGGTGCC.

In some embodiments, the polynucleotide encodes a 3-epimerase polypeptide. In some embodiments, the polynucleotide encodes a D-psicose 3-epimerase polypeptide.

As used herein, the term “3-epimerase enzyme refers to any peptide, polypeptide, or a protein, capable of catalyzing the isomerization of C₃ position of ketose monosaccharides.

According to some embodiments, there is provided an artificial nucleic acid molecule comprising the polynucleotide disclosed herein.

In some embodiments, the artificial nucleic acid comprises an artificial vector.

In some embodiments, the artificial vector comprises a plasmid. In some embodiments, the artificial vector comprises or is an Agrobacterium comprising the artificial nucleic acid molecule. In some embodiments, the artificial vector is an expression vector. In some embodiments, the artificial vector is a plant expression vector. In some embodiments, the artificial vector is a bacterial expression vector. In some embodiments, the artificial vector is a fungal expression vector. In some embodiments, the artificial vector is a yeast expression vector. In some embodiments, the artificial vector is for use in expressing a 3-epimease encoding nucleic acid sequence as disclosed herein. In some embodiments, the artificial vector is for use in expressing a D-psicose 3-epimerase encoding nucleic acid sequence as disclosed herein. In some embodiments, the artificial vector is for use in heterologous expression of a 3-epimerase encoding nucleic acid sequence as disclosed herein in a cell, a tissue, or an organism. In some embodiments, the artificial vector is for use in heterologous expression of a D-psicose 3-epimerase encoding nucleic acid sequence as disclosed herein in a cell, a tissue, or an organism. In some embodiments, the artificial vector is for use in producing or the production of a D-psicose in a cell, a tissue, or an organism.

Expressing of a polynucleotide within a cell is well known to one skilled in the art. It can be carried out, among many methods, by transfection, viral infection, or direct alteration of the cell's genome. In some embodiments, the polynucleotide is in an expression vector such as plasmid or viral vector. A vector nucleic acid sequence generally contains at least an origin of replication for propagation in a cell and optionally additional elements, such as a heterologous polynucleotide sequence, expression control element (e.g., a promoter, enhancer), selectable marker (e.g., antibiotic resistance), poly-Adenine sequence.

The vector may be a DNA plasmid delivered via non-viral methods or via viral methods. The viral vector may be a retroviral vector, a herpesviral vector, an adenoviral vector, an adeno-associated viral vector, a virgaviridae viral vector, or a poxviral vector. The barley stripe mosaic virus (BSMV), the tobacco rattle virus and the cabbage leaf curl geminivirus (CbLCV) may also be used. The promoters may be active in plant cells. The promoters may be a viral promoter.

In some embodiments, the polynucleotide as disclosed herein is operably linked to a promoter. The term “operably linked” is intended to mean that the nucleotide sequence of interest is linked to the regulatory element or elements in a manner that allows for expression of the nucleotide sequence (e.g., in an in vitro transcription/translation system or in a host cell when the vector is introduced into the host cell). In some embodiments, the promoter is operably linked to the polynucleotide of the invention. In some embodiments, the promoter is a heterologous promoter. In some embodiments, the promoter is the endogenous promoter.

As used herein, the term “heterologous” encompasses any case wherein the polynucleotide encoding the gene of interest (e.g., a polypeptide of the invention as disclosed herein) is operably linked to a promoter other than the endogenous promoter of the gene of interest. In some embodiments, a heterologous promoter and a polynucleotide encoding the polypeptide of the invention are not derived from the same organism, are not derived from the same gene or genomic location, or both.

In some embodiments, the vector is introduced into the cell by standard methods including electroporation (e.g., as described in From et al., Proc. Natl. Acad. Sci. USA 82, 5824 (1985)), heat shock, infection by viral vectors, high velocity ballistic penetration by small particles with the nucleic acid either within the matrix of small beads or particles, or on the surface (Klein et al., Nature 327. 70-73 (1987)), such as biolistic use of coated particles, and needle-like particles, Agrobacterium Ti plasmids and/or the like. The term “promoter” as used herein refers to a group of transcriptional control modules that are clustered around the initiation site for an RNA polymerase i.e., RNA polymerase II. Promoters are composed of discrete functional modules, each consisting of approximately 7-20 bp of DNA, and containing one or more recognition sites for transcriptional activator or repressor proteins. The promoter may extend upstream or downstream of the transcriptional start site and may be any size ranging from a few base pairs to several kilo-bases.

In some embodiments, the polynucleotide is transcribed by RNA polymerase II (RNAP II and Pol II). RNAP II is an enzyme found in eukaryotic cells, known to catalyze the transcription of DNA to synthesize precursors of mRNA and most snRNA and microRNA.

Examples of suitable promoters for directing transcription of the nucleic acid constructs of the present invention in a bacterial host cell are the promoters obtained from the Bacillus amyloliquefaciens alpha-amylase gene (amyQ), Bacillus licheniformis alpha-amylase gene (amyL), Bacillus licheniformis penicillinase gene (penP), Bacillus stearothermophilus maltogenic amylase gene (amyM), Bacillus subtilis levansucrase gene (sacB), Bacillus subtilis xylA and xylB genes, Bacillus thuringiensis crylllA gene (Agaisse and Lereclus, 1994, Molecular Microbiology 13:97-107), E. coli lac operon, E. coli trc promoter (Egon et al., 1988, Gene 69: 301-315), Streptomyces coelicolor agarase gene (dagA), and prokaryotic beta-lactamase gene (Villa-Kamaroff et al., 1978, Proc. Natl. Acad. Sci. USA 75: 3727-3731), as well as the tac promoter (DeBoer et al., 1983, Proc. Natl. Acad. Sci. USA 80: 21-25). Further promoters are described in “Useful proteins from recombinant bacteria” in Gilbert et al., 1980, Scientific American 242: 74-94; and in Sambrook et al., 1989, supra. Examples of tandem promoters are disclosed in WO 99/43835.

In some embodiments, a plant expression vector is used. In one embodiment, the expression of a polypeptide coding sequence is driven by a number of promoters. In some embodiments, viral promoters such as the 35S RNA and 19S RNA promoters of CaMV [Brisson et al., Nature 310:511-514 (1984)], or the coat protein promoter to TMV [Takamatsu et al., EMBO J. 6:307-311 (1987)] are used. In another embodiment, plant promoters are used such as, for example, the small subunit of RUBISCO [Coruzzi et al., EMBO J. 3: 1671-1680 (1984); and Brogli et al., Science 224:838-843 (1984)] or heat shock promoters, e.g., soybean hsp17.5-E or hsp17.3-B [Gurley et al., Mol. Cell. Biol. 6:559-565 (1986)]. In one embodiment, constructs are introduced into plant cells using Ti plasmid, Ri plasmid, plant viral vectors, direct DNA transformation, microinjection, electroporation and other techniques well known to the skilled artisan. See, for example, Weissbach & Weissbach [Methods for Plant Molecular Biology, Academic Press, NY, Section VIII, pp 421-463 (1988)]. Other expression systems such as insects and mammalian host cell systems, which are well known in the art, can also be used by the present invention.

In some embodiments, expression vectors containing regulatory elements from eukaryotic viruses such as retroviruses are used by the present invention. For example, SV40 vectors may include pSVT7 and pMT2. In some embodiments, vectors derived from bovine papilloma virus include pBV-IMTHA, and vectors derived from Epstein Bar virus include pHEBO, and p205. Other exemplary vectors include pMSG, pAV009/A+, pMTO10/A+, pMAMneo-5, baculovirus pDSVE, and any other vector allowing expression of proteins under the direction of the SV-40 early promoter, SV-40 later promoter, metallothionein promoter, murine mammary tumor virus promoter, Rous sarcoma virus promoter, polyhedrin promoter, or other promoters shown effective for expression in eukaryotic cells.

In some embodiments, recombinant viral vectors, which offer advantages such as systemic infection and targeting specificity, are used for in vivo expression. In one embodiment, systemic infection is inherent in the life cycle of, for example, the retrovirus and is the process by which a single infected cell produces many progeny virions that infect neighboring cells. In one embodiment, the result is that a large area becomes rapidly infected, most of which was not initially infected by the original viral particles. In one embodiment, viral vectors are produced that are unable to spread systemically. In one embodiment, this characteristic can be useful if the desired purpose is to introduce a specified gene into only a localized number of targeted cells.

In some embodiments, plant viral vectors are used. In some embodiments, a wild-type virus is used. In some embodiments, a deconstructed virus such as are known in the art is used. In some embodiments, Agrobacterium is used to introduce the vector of the invention into a virus.

Various methods can be used to introduce the expression vector of the present invention into cells. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504-512, 1986] and include, for example, stable or transient transfection, lipofection, electroporation, Agrobacterium Ti plasmids and infection with recombinant viral vectors. In addition, see U.S. Pat. Nos. 5,464,764 and 5,487,992 for positive-negative selection methods.

It will be appreciated that other than containing the necessary elements for the transcription and translation of the inserted coding sequence (encoding the polypeptide), the expression construct of the present invention can also include sequences engineered to optimize stability, production, purification, yield, or activity of the expressed polypeptide.

In some embodiments, the artificial vector comprises a polynucleotide encoding a protein comprising an amino acid sequence as described herein.

According to some embodiments, there is provided a transgenic cell or a transfected cell comprising: (a) the polynucleotide disclosed herein; (b) the artificial nucleic acid molecule disclosed herein; (c) the plasmid or Agrobacterium disclosed herein; (d) the protein disclosed herein; or any combination thereof.

As used herein, the terms “transformed cell”, “transgenic cell” or “transfected cell” refer to any cell that has undergone human manipulation so as to include an exogenous polynucleotide. In some embodiments, the exogenous polynucleotide is integrated into the genome of the cell (e.g., manipulation on the genomic or gene level). In some embodiments, the exogenous polynucleotide is not integrated into the genome of the cell. In some embodiments, the transformed cell, transgenic cell or a transfected cell has had exogenous polynucleotide, such as an isolated DNA molecule as disclosed herein, introduced into it. In some embodiments, a transformed cell, a transgenic cell or a transfected cell comprises a cell that has an artificial vector introduced into it. In some embodiments, a transgenic cell or a transfected cell is a cell which has undergone genome mutation or modification. In some embodiments, a transgenic cell or a transfected cell is a cell that has undergone CRISPR genome editing. In some embodiments, a transgenic cell or a transfected cell is a cell that has undergone targeted mutation of at least one base pair of its genome. In some embodiments, the exogenous polynucleotide (e.g., the isolated DNA molecule disclosed herein) or vector is stably integrated into the cell. In some embodiments, the transgenic cell or the transfected cell expresses a polynucleotide of the invention. In some embodiments, the transgenic cell or the transfected cell expresses a vector of the invention. In some embodiments, the transgenic cell or the transfected cell expresses a protein of the invention. In some embodiments, the transgenic cell or the transfected cell, is a cell that is devoid of a polynucleotide of the invention that has been transformed or genetically modified to include the polynucleotide of the invention. In some embodiments, CRISPR technology is used to modify the genome of the cell, as described herein.

In some embodiments, the cell is a unicellular organism, a cell of a multicellular organism, and a cell in a culture.

In some embodiments, a unicellular organism comprises a fungus or a bacterium.

In some embodiments, the fungus is a yeast cell.

In some embodiments, the transformed cell, transgenic cell, or transfected cell is selected from: Bacillus licheniformis, Saccharomyces cerevisiae, Schizosaccharomyces pombe, Pseudomonas putida, Pichia sp., Aspergillus sp., Trichoderma reesei, Corynebacterium glutamicum, E. coli, or B. subtilis.

In some embodiments, the cell is a plant cell. In some embodiments, the cell comprises a plant cell line. In some embodiments, the cell is an insect cell. In some embodiments, the cell comprises an insect cell line.

Types of plant and/or insect cell lines suitable for transformation and/or heterologous expression are common and would be apparent to one of ordinary skill in the art. Non-limiting examples of insect cell lines include, but are not limited to, Sf-9 cells, SR+ Schneider cells, S2 cells, and others.

According to some embodiments, there is provided an extract derived from a transgenic cell or a transfected cell disclosed herein, or any fraction thereof.

In some embodiments, the extract comprises the polynucleotide of the invention, an isolated DNA molecule as disclosed herein, a protein as disclosed herein, or any combination thereof.

According to some embodiments, there is provided a homogenate, lysate, extract, derived from a transgenic cell or a transfected cell disclosed herein, any combination thereof, or any fraction thereof.

Methods and/or means for extracting, lysing, homogenizing, fractionating, or any combination thereof, a cell or a culture of same, are common and would be apparent to one of ordinary skill in the art of cell biology and biochemistry. Non-limiting examples include, but are not limited to, pressure lysis (e.g., such as using a French press), enzymatic lysis, soluble-insoluble phase separation (such for obtaining a supernatant and a pellet), detergent-based lysis, solvent (e.g., polar, or nonpolar solvent), liquid chromatography mass spectrometry, or others.

According to another aspect, there is provided a transgenic plant, a transgenic plant tissue or a plant part, comprising: (a) a nucleic acid sequence of the invention; (b) the plasmid or expression vector disclosed herein; (c) the polypeptide of the invention; (d) the transgenic cell or transfected cell disclosed herein; or (e) any combination of (a) to (d).

According to some embodiments, there is provided a composition comprising the herein disclosed: (a) a polypeptide of the invention; (b) a polynucleotide of the invention (for example, an isolated DNA molecule); (c) a plasmid or an expression vector; (d) a transgenic cell as described herein; (e) an extract of the transgenic cell or transfected cell; or (f) any combination of (a) to (e), and an acceptable carrier.

In some embodiments, the composition further comprises a divalent metal cation.

In some embodiments, the divalent cation is selected from: Co²⁺, Mg²⁺, Mn²⁺, Mo²⁺, or Ni².

In some embodiments, the acceptable carrier is or comprises an aqueous solvent.

In some embodiments, the polypeptide is bound to a solid support. In some embodiments, the polypeptide within the composition of the invention is bound (e.g., covalently or non-covalently) to the solid support.

In some embodiments, bound is via a covalent bond. In some embodiments, the polypeptide is covalently bound to a solid support. In some embodiments, bound comprises directly or indirectly bound. In some embodiments, indirectly is via a chemical linker or an agent. In some embodiments, bound is via a non-covalent interaction (e.g., electrostatic interaction).

In some embodiments, the composition further comprises a chemical agent capable of binding both the polypeptide and the solid support. In some embodiments, the composition further comprises a chemical agent capable of binding to the polypeptide and to the solid support.

In some embodiments, the composition further comprises fructose, D-psicose, or both.

As used herein, the term “carrier”, “excipient”, or “adjuvant” refers to any component of a composition, e.g., pharmaceutical or nutraceutical, that is not the active agent. As used herein, the term “acceptable carrier” refers to non-toxic, inert, solid, semi-solid or liquid filler, diluent, encapsulating material, formulation auxiliary of any type, or simply a sterile aqueous medium, such as saline. Some examples of the materials that can serve as acceptable carriers are sugars, such as lactose, glucose and sucrose, starches such as corn starch and potato starch, cellulose and its derivatives such as sodium carboxymethyl cellulose, ethyl cellulose and cellulose acetate; powdered tragacanth; malt, gelatin, talc; excipients such as cocoa butter and suppository waxes; oils such as peanut oil, cottonseed oil, safflower oil, sesame oil, olive oil, corn oil and soybean oil; glycols, such as propylene glycol, polyols such as glycerin, sorbitol, mannitol and polyethylene glycol; esters such as ethyl oleate and ethyl laurate, agar; buffering agents such as magnesium hydroxide and aluminum hydroxide; alginic acid; pyrogen-free water; isotonic saline, Ringer's solution; ethyl alcohol and phosphate buffer solutions, as well as other non-toxic compatible substances used in pharmaceutical formulations. Some non-limiting examples of substances which can serve as a carrier herein include sugar, starch, cellulose and its derivatives, powered tragacanth, malt, gelatin, talc, stearic acid, magnesium stearate, calcium sulfate, vegetable oils, polyols, alginic acid, pyrogen-free water, isotonic saline, phosphate buffer solutions, cocoa butter (suppository base), emulsifier (e.g. carbomer, hydroxypropyl cellulose, sodium lauryl sulfate) as well as other non-toxic pharmaceutically compatible substances used in other pharmaceutical formulations. Wetting agents and lubricants such as sodium lauryl sulfate, as well as coloring agents, flavoring agents, excipients, stabilizers, antioxidants, and preservatives may also be present. Any non-toxic, inert, and effective carrier may be used to formulate the compositions contemplated herein.

The carrier may comprise, in total, from about 0.1% to about 99.99999% by weight of the compositions presented herein.

### Methods of Synthesis

According to some embodiments, there is provided a method for synthesizing a polypeptide having 3-epimerase activity, comprising the steps: (a) providing a cell comprising a plasmid or an expression vector comprising a nucleic acid sequence having at least 70% sequence identity to a nucleic acid sequence selected from: SEQ ID Nos.: 17-32; and (b) culturing the cell from step (a) such that a polypeptide encoded by the plasmid or the expression vector is expressed, thereby synthesizing the polypeptide.

In some embodiments, the method further comprises at least one step selected from: recovering the polypeptide, at least partially purifying the polypeptide, immobilizing the polypeptide, and any combination thereof.

In some embodiments, immobilizing is to a solid support. In some embodiments, the polypeptide comprises an amino acid sequence having increased binding affinity to the solid support. In some embodiments, the polypeptide comprises a chemical moiety having reactivity to the solid support.

In some embodiments, the method comprises culturing a transgenic cell or a transfected cell in a medium and extracting the transgenic cell or the transfected cell.

In some embodiments, the method comprises the steps: (a) culturing a transgenic cell or a transfected cell in a medium; and (b) extracting the transgenic cell or the transfected cell, thereby obtaining an extract from the transgenic cell or the transfected cell.

In some embodiments, the transgenic cell or the transfected cell comprises a plasmid or an expression vector comprising a nucleic acid sequence having at least 70%, at least 75%, at least 80%, at least 85%, at least 89%, at least 92%, at least 95%, or at least 99% homology or identity to any one of SEQ ID Nos.: 17-32, or any value and range therebetween. Each possibility represents a separate embodiment of the invention.

In some embodiments, the transgenic cell or the transfected cell comprises the polynucleotide of the invention or a plurality thereof, as disclosed herein.

In some embodiments, the transgenic cell or the transfected cell comprises the polynucleotide, the plasmid or the expression vector comprising same, as disclosed herein, or any combination thereof.

In some embodiments, the cell is a transformed cell, a transgenic cell, or a cell transfected with a polynucleotide as disclosed herein.

In some embodiments, the cell is a prokaryote cell or a eukaryote cell.

In some embodiments, the cell is a transformed cell, a transgenic cell, or a cell transfected with the herein disclosed plasmid or expression vector.

In some embodiments, the method further comprises a step preceding step (a), comprising introducing or transfecting the cell with the plasmid or expression vector, disclosed herein.

Method for introducing or transfecting a cell with an artificial nucleic acid molecule or vector are common and would be apparent to one of ordinary skill in the art.

In some embodiments, introducing or transfecting comprises transferring a plasmid or an expression vector comprising the polynucleotide disclosed herein into a cell; or modifying the genome of a cell to include the polynucleotide disclosed herein. In some embodiments, the transferring comprises transfection. In some embodiments, the transferring comprises transformation. In some embodiments, the transferring comprises lipofection. In some embodiments, the transferring comprises nucleofection. In some embodiments, the transferring comprises viral infection.

As used herein, the terms “transfecting” and “introducing” are interchangeable.

In some embodiments, introducing does not involve virus or viral derived particles, and/or methodologies.

According to some embodiments, there is provided a method for producing D-psicose comprising the steps: (a) contacting the composition disclosed herein, with an effective amount of fructose, thereby obtaining a reaction mixture; and (b) subjecting the reaction mixture to conditions suitable for at least partial conversion of the fructose to D-psicose, thereby producing D-psicose. In some embodiments, step a is performed in an aqueous solution (also referred to herein as “the reaction mixture”). In some embodiments, the aqueous solution has a pH between 3 and 9, between 5 and 9, between 5.5 and 9, between 5.5 and 8, between 5.5 and 7.5, between 6 and 9, between 6 and 8, between 6 and 7.5, including any range between.

In some embodiments, a w/w concentration of the polypeptide within the reaction mixture is between 0.1 and 10, between 0.1 and 1, between 0.2 and 1, between 0.2 and 0.5 g/L, including any range between.

As used herein, the term “partial” refers to % ranging from 0.01 to 99%.

In one embodiment, the conditions comprise: (i) a temperature between 25° C. and 75° C., between 30° C. and 70° C.; between 40° C. and 60° C., between 45° C. and 75° C., between 45° C. and 70° C., between 45° C. and 60° C., between 40° C. and 55° C., between about 40° C. and about 50° C.; (ii) a pH of between 3 and 9, between 5 and 9, between 5.5 and 9, between 5.5 and 8, between 5.5 and 7.5, between 6 and 9, between 6 and 8, between 6 and 7.5, or both (i) and (ii).

In some embodiments, subjecting comprises contacting the reaction mixture with a divalent metal cation, as described herein.

In some embodiments, the divalent metal cation is present in the reaction mixture in a concentration ranging from 0.1 mM to 10 mM.

In some embodiments, the method further comprises a step comprising isolating the D-psicose from the reaction mixture.

In some embodiments, isolating comprises: extraction, precipitation, membrane filtration, or any combination thereof.

According to some embodiments, the method comprises contacting a fructose with any one of: the polypeptide of the invention or a functional analog thereof, as described herein, a cell comprising same, or an extract derived therefrom, thereby producing D-psicose.

In some embodiments, the culturing comprises supplementing the cell with an effective amount of a fructose. In some embodiments, the supplementing is via the growth or culture medium wherein the cell is cultured.

In some embodiments, the contacting is in a cell-free system.

Types of suitable cell-free systems utilizing any one of: the polynucleotide of the invention or a plurality thereof, as disclosed herein, and the protein of the invention, or a plurality thereof, would be apparent to one of ordinary skill in the art.

In some embodiments, the method further comprises a step preceding step (b), comprising separating the cultured transgenic cell or the cultured transfected cell from the medium.

Method for separating cell from a medium are common and may include, but not limited to, centrifugation, ultracentrifugation, or other, as would be apparent to one of ordinary skill in the art.

According to some embodiments, there is provided an extract of a transformed cell, a transgenic cell, or a transfected cell obtained according to the herein disclosed method.

In some embodiments, the extract comprises an extract of the medium wherein the transformed cell, transgenic cell, or transfected cell is cultured.

In some embodiments, the extract comprises an extract of the transformed cell, transgenic cell, or transfected cell, including any fraction thereof.

In some embodiments, the extract comprises a lysate, a homogenate, a polar extract, a non-polar extract, any fraction thereof, or any combination thereof of any one of the transformed cell, transgenic cell, or transfected cell. In some embodiments, the extract further comprises medium wherein the transformed cell, transgenic cell, or transfected cell is cultured, or a carrier wherein the carrier is as described herein.

According to some embodiments, there is provided a medium or a portion thereof separated from a cultured transgenic cell or a cultured transfected cell, obtained according to the herein disclosed method.

According to some embodiments, there is provided a composition comprising: (a) the extract disclosed herein; (b) the medium disclosed herein or a portion thereof; or (c) any combination of (a) and (b), and an acceptable carrier, as described herein.

In some embodiments, a portion comprises a fraction or a plurality thereof.

### General

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

As used herein, the term “about” when combined with a value refers to plus and minus 10% of the reference value. For example, a length of about 1,000 nanometers (nm) refers to a length of 1,000 nm±100 nm.

It is noted that as used herein and in the appended claims, the singular forms “a,” “an,” and “the” include plural referents unless the context clearly dictates otherwise. Thus, for example, reference to “a polynucleotide” includes a plurality of such polynucleotides and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements or use of a “negative” limitation.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B”.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

Various embodiments and aspects of the present invention as delineated hereinabove and as claimed in the claims section below find experimental support in the following examples.

### EXAMPLES

Generally, the nomenclature used herein, and the laboratory procedures utilized in the present invention include molecular, biochemical, microbiological, and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells-A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, CT (1994); Mishell and Shiigi (eds), “Strategies for Protein Purification and Characterization—A Laboratory Course Manual” CSHL Press (1996); all of which are incorporated by reference. Other general references are provided throughout this document.

### Materials and Methods

### Growth Media Preparation

Growth of the recombinant cells was performed as described in the art. See for example Chang-Su Park et. al., 2016, J Bioscience Bioengineering vol. 121 (2) and WO2018116266. Briefly, transformed clones (carrying the expression vector) were grown on Terrific Broth (TB) medium or defined media containing kanamycin for overnight at 37° C. Overnight culture was re-inoculated and grown to 0.6-0.9 OD6oo in TB (Kan+). Expression of the polypeptides of the present invention in the transformed cells was induced by addition of IPTG during fermentation process. The polypeptides were extracted and separated from the cells by lysis, centrifugation, resuspension and mounting on a column. Media and buffer compositions and concentration are as known in the art and may contain for example:

### Terrific Broth (TB)

Formula per 1 Liter in water: Tryptone (12.0 g); yeast extract (24.0 g); and glycerol 100% (5 ml) in 900 ml, autoclave separately.

Dipotassium Phosphate [K₂HPO₄] (12.54 g); and monopotassium phosphate [KH₂PO₄] (2.39 g) in 100 ml, autoclave separately.

### Lysis Buffer

NaCl [100-500 mM]; 1 Tris [10-100 mM]; Tween20 [0.05-0.25%]; DTT [0.1-10 mM]; and PMSF 1-50 mM; pH 6-9.

### His-Tag Ni Buffer

Tris [10-100 mM]; NaCl [100-500 mM], and imidazole [25-500 mM]; pH 6-9.

### Dialysis Storage Buffer)

NaCl [100-500 mM]; Tris-HCl [10-100 mM]; DTT [0.1-25 mM].

Kanamycin stock solution [10-30 mg ml]

To be later used as 1:1,000.

### Day 0

Single colonies were grown overnight at 37° C. Five (5) ml of TB medium supplemented with 5 μl kanamycin. Ten (10) ml of the growth medium w/o bacteria were kept and later used as background.

### Day 1

From the overnight (O.N.) culture, 5 ml were inoculated into 500 mL TB broth containing kanamycin [10-30 mg/ml], in a 2.5 L Erlenmeyer flask, and grown at 37° C. under 250-500 rpm shaking.

Culture overpassed OD₆₀₀ of approx. 0.6-0.9. A sample of volume equivalent to 1 O.D. was collected into an Eppendorf tube, and used as time zero (T0).

Protein expression was induced with [0.1-10 mM] IPTG.

Cells were grown overnight at 15-37° C.

### Day 2

Transfer the TON culture into centrifuge tube, centrifuge at 8,000 rpm for 10 min at 4° C.

All stages onwards were performed under cooling conditions.

Supernatant was decanted and pellet was resuspended with 20 ml of cold 0.9% NaCl, followed by centrifugation at 14,000 rpm for 10 min at 4° C.

Samples were resuspended in 40-80 ml lysis buffer and sonicated 3-4 times.

Thereafter, samples were centrifuged at 14,000 rpm, at 4° C., 20 min. Before column loading, the liquid was filtered—using a 0.44 μm filter.

### Results

Exemplary polypeptides of the current invention have been isolated and purified on an AKTA purifier, as described hereinbelow:

### Ni-sepharose FF Column Batch Record—Exp_20: B1_C3_N58 (SEQ ID NO: 11) (FIG. l)

| | Solution | Column Id |
|---|---|---|
| 1 | Buffer A: Tris 10-100 mM, 100- | Ni-trap |
| | 500 mM NaCl (pH 6.0-9.0) | |
| 3 | Buffer B: Buffer A + 500 mM imidazole | Volume: 1-100 ml |
| 4 | Strip: Buffer B | Chromatography |
| 5 | Regeneration: 0.5M NaOH | |
| 6 | Storage: 20% ethanol | AKTA start |

### Preparations

Load fraction: Exp_20: B1_C3_N58 (F003)
   Column equilibration: Flow rate: 1-5 ml/min
Tris 10-100 mM, 100-500 mM NaCl (pH 6.0-9.0)
   Column load: Flow rate: 1-5 ml/min
Load fraction loaded Volume.
   Column wash: Buffer A: Tris 10-100 mM, 100-500 mM NaCl (pH 6.0-9.0)+10-125 mM imsidazole
   Elution: Buffer A: Tris 10-100 mM, 100-500 mM NaCl (pH 6.0-9.0)+125-450 mM imidazole

### Strip: Buffer B

Regeneration: Flow rate: 1-5 ml/min
a. 6CV 0.5 M NaOH
b. 10CV RO
c. 4CV 20% ethanol

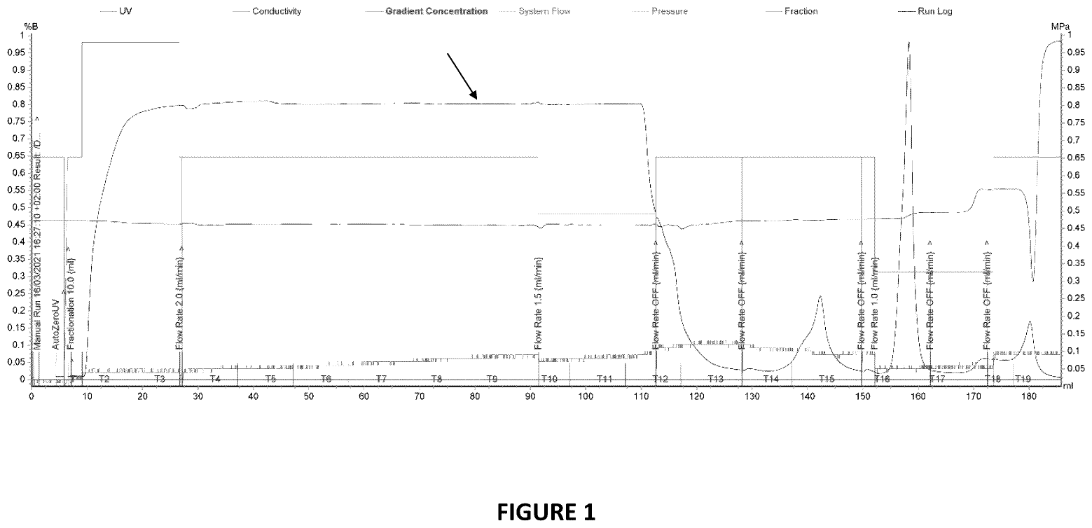

As depicted in FIG. 1., the solid line (marked by an arrow) indicates absorbance at 280 nm by aromatic amino acid residues of the protein. Numerals 1-5 depict different stages of separation: 1-Loading of the sample (a lysate); 2-Wash, separation, of unbound proteins with Buffer A; 3-Wash, separation, of low affinity non-specifically bound proteins with Buffer A with 25 mM imidazole; 4-Elution, separation, of high-affinity bound proteins (including his tagged proteins) with Buffer A with 125-450 mM imidazole, and; 5-Strip using Buffer B for separation of high-affinity bound proteins (his-tagged proteins) followed by a denaturalizing wash using 0.5 M NaOH, to clean and regenerate the column.

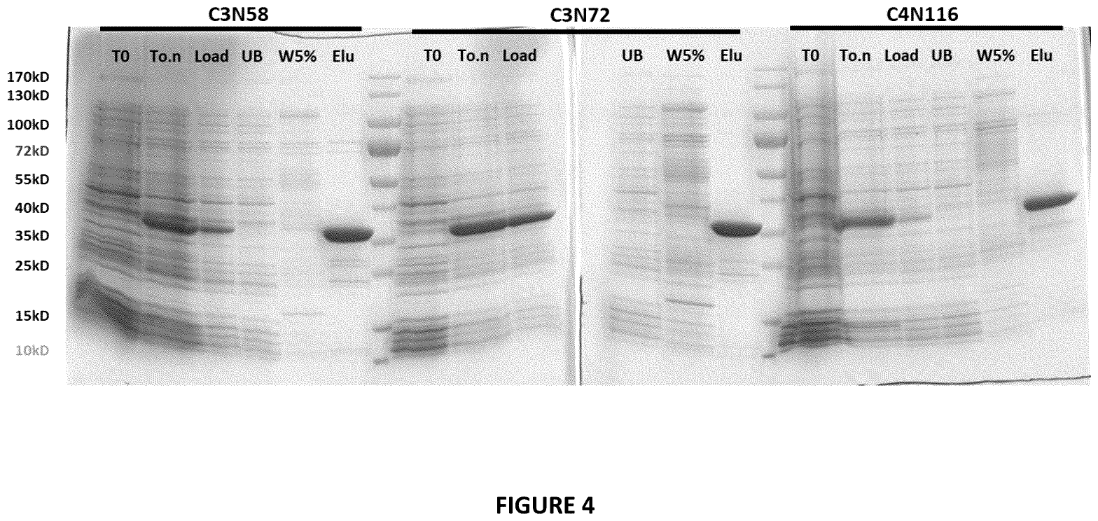

The different protein fractions were resolved on SDS-PAGE and stain with Coomassie blue stain (FIG. 4): T₀-indicates cell lysate that was collected before IPTG induction of plasmid gene expression; T_{o.n}-indicates cell lysate that was collected after over 12 hr of IPTG induction; Load-indicates the cells soluble fraction that was loaded on the nickel column; UB-indicates the fraction that passed the column and contains all the unbound proteins that did not bind to the nickel resin; W5%—indicates column wash with 10-125 mM imidazole and contains all the proteins that bind weakly (non-specific binding) to the nickel resin; EL-elution of proteins that bind strongly (specific binding) to the nickel resin was achieved by washing the column with 125-450 mM imidazole.

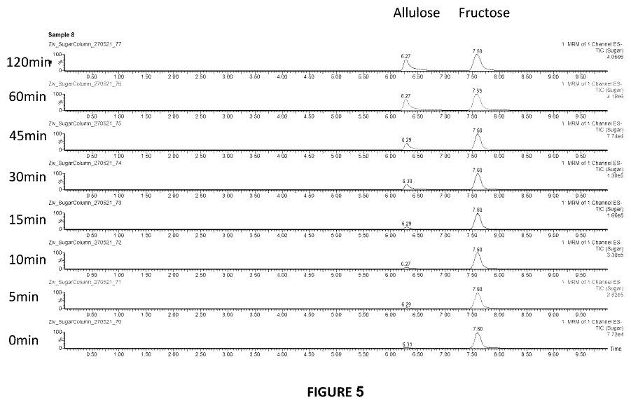

To validate the efficiency of allulose formation from D-fructose, 10-100 mM D-fructose was mixed with 2-200 μg of a polypeptide of the invention in phosphate buffer (pH 6-9) containing 0.2-20 mM MgCl₂. The reaction was incubated for 0-120 min at 37° C. Conversion of fructose to allulose was assessed via HPLC chromatography using a Luna® Omega 3 μm SUGAR 100A LC column 250×4.6 mm (Phenomenex). The crude reaction mixture was analyzed by running an isocratic flow of the mobile phase composition (DDW: ACN). Allulose retention time (RT) was at 6.3 min and that of fructose was at 7.6 min (FIG. 5). As represented in FIG. 5, 10 min after the reaction onset a significant amount of allulose could be detected in the reaction mixture.

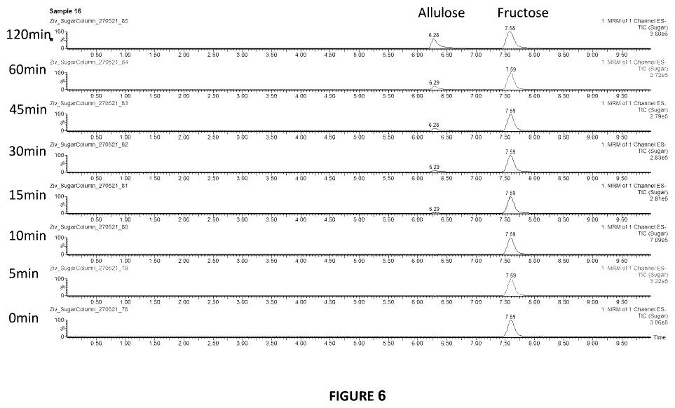

A repeating experiment was performed, i.e., examination of allulose production from D-fructose with the C3_N72 polypeptide of the present invention (SEQ ID No.: 13), but this time using moderately acidic (pH 5-6) conditions (FIG. 6). As represented by FIG. 6, a significant amount of allulose was detected 30 min after the reaction onset.

Exemplary polypeptides of the invention (SEQ ID Nos.: 4, 9, 10, 11, 12, 13, 14, and 16) showed a significant D-fructose to allulose catalytic activity. To this end, from the above polypeptides, 3 polypeptides (SEQ ID Nos.: 11, 12, and 13) showed the greatest catalytic activity at a pH range between about 5.5 and about 7. It is further postulated that the workable pH range of the polypeptides of the present invention is between about 5 and about 9.

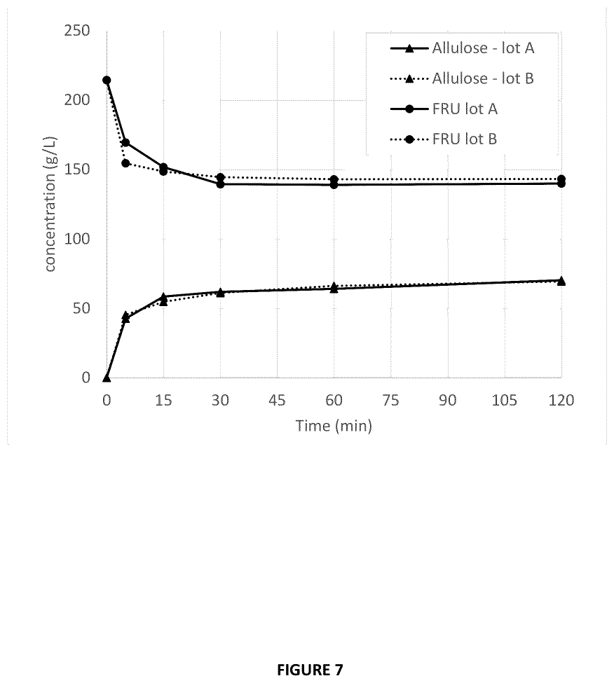

A pilot experiment for the assessment of D-fructose to allulose conversion by utilizing exemplary polypeptides of the present invention (SEQ ID Nos.: 11 and 13) has been performed as described hereinbelow. The results of this experiment are represented in FIG. 7. As shown in FIG. 7, the tested polypeptide induced that about 25% conversion (about 60 g fructose has been converted to an equivalent amount of allulose) only 15 min after the reaction onset, resulting in the formation of about 60 g/L allulose.

In brief, a feeding solution (total volume of about 1L) containing 200 g D-Fructose, K₂HPO₄ (3.99 g), KH₂PO₄ (2.72 g), MgCl₂ (0.41 g) together with an exemplary polypeptide of the invention (0.42 g/L) has been introduced into a reactor. The reaction has been performed at about 50° C. under gentle stirring. Conversion rate has been determined by HPLC, as described hereinabove.

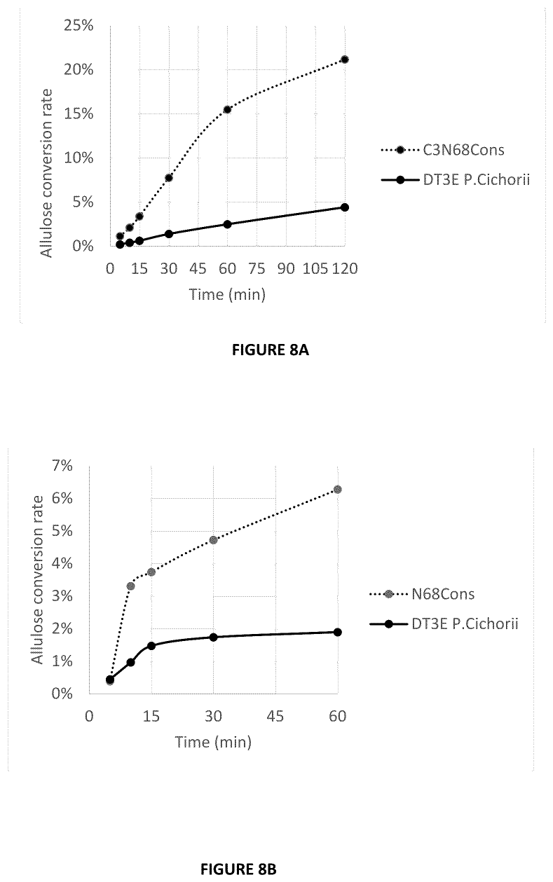

Moreover, the inventors demonstrated superior conversion rate of the tested polypeptides of the invention, as compared to a control (D-tagatose-3-epimerase from Pseudomonas Chicorri), having a sequence identical with the commercial enzyme from ADM. As demonstrated in FIGS. 8A-B, an exemplary polypeptide of the invention (SEQ ID No.: 12) showed a significantly higher conversion rate (about 4 to 6 times higher rate), as compared to the control at pH 6 and 7. As demonstrated by FIG. 8A, at pH 7, the tested polypeptide exhibited more than 20% conversion rate after 2 h reaction time. Additional polypeptides of the invention (e.g., SEQ ID Nos.: 11 and 13) demonstrated even higher catalytic performance (data not shown).

Although the invention has been described in conjunction with specific embodiments thereof, it is evident that many alternatives, modifications, and variations will be apparent to those skilled in the art. Accordingly, it is intended to embrace all such alternatives, modifications and variations that fall within the spirit and broad scope of the appended claims.

1. A polypeptide comprising an amino acid sequence selected from the group consisting of:
(i) SEQ ID Nos: 1-12 and 14-16, or a functional analog having at least 88% sequence homology thereto; and
(ii) SEQ ID No 13, or a functional analog having at least 92% sequence homology thereto.

2. The polypeptide of claim 1, characterized by having a 3-epimerase activity.

3. The polypeptide of claim 2, wherein said 3-epimerase activity comprises D-psicose 3-epimerase activity, and wherein the polypeptide comprises an amino acid sequence selected from SEQ ID Nos.: 4, 9, 10, 11, 12, 13, 14, and 16.

4. The polypeptide of claim 1, being an isolated polypeptide or a synthetic polypeptide, and wherein the polypeptide comprises an amino acid sequence selected from SEQ ID Nos.: 11, 12, and 13.

5. A polynucleotide encoding the polypeptide of claim 1.

6. The polynucleotide of claim 5, comprising a nucleic acid sequence selected from the group consisting of: SEQ ID Nos: 17-32; and wherein said polynucleotide is an isolated or an artificial polynucleotide.

7. (canceled)

8. (canceled)

9. A transgenic, transformed, or a transfected cell comprising:
a. the polynucleotide of claim 5.

10. The transgenic, transformed, or the transfected cell of claim 9, being any one of: a unicellular organism, a cell of a multicellular organism, and a cell in a culture, and optionally wherein said unicellular organism comprises a fungus or a bacterium, optionally wherein said fungus is a yeast cell, or both.

11. (canceled)

12. (canceled)

13. The transgenic cell, transformed, or the transfected cell of claim 9, wherein said transgenic cell or said transfected cell is selected from the group consisting of: Bacillus licheniformis, Saccharomyces cerevisiae, Schizosaccharomyces pombe, Pseudomonas putida, Pichia sp., Aspergillus sp., Trichoderma reesei, Corynebacterium glutamicum, E. coli and B. subtilis, or wherein said a cell of a multicellular organism comprises a plant cell.

14. (canceled)

15. An extract derived from the transgenic cell or the transfected cell of claim 9, or any fraction thereof, and optionally wherein said extract comprises said polynucleotide.

16. (canceled)

17. A transgenic plant, a transgenic plant tissue or a plant part, comprising:
a. the polynucleotide of claim 5.

18. A composition comprising the polypeptide of claim 1, and an acceptable carrier, and optionally wherein any one of: (i) said composition further comprises a divalent metal cation, and optionally wherein said divalent cation is selected from the group consisting of: Co²⁺, Mg²⁺, Mn²⁺, Mo²⁺, Ni², and any combination thereof; (ii) said composition further comprises an aqueous solvent; (iii) said polypeptide is bound to a solid support, and optionally wherein said bound is via a covalent bond or a non-covalent interaction, said composition further comprises a chemical agent capable of binding both said polypeptide and said solid support, or both; (iv) said composition further comprises fructose, D-psicose, or both; and (v) any combination of (i) to (iv).

19. (canceled)

20. (canceled)

21. (canceled)

22. (canceled)

23. (canceled)

24. (canceled)

25. (canceled)

26. A method for synthesizing a polypeptide having 3-epimerase activity, comprising the steps:
a. providing a cell comprising a plasmid or an expression vector comprising a nucleic acid sequence having at least 70% sequence identity to a nucleic acid sequence selected from the group consisting of: SEQ ID Nos: 17-32; and
b. culturing said cell from step (a) such that a polypeptide encoded by said plasmid or an expression vector is expressed,
thereby synthesizing the polypeptide.

27. The method of claim 26, wherein any one of: (i) said method further comprises at least one step selected from the group consisting of: recovering said polypeptide, at least partially purifying the polypeptide, immobilizing said polypeptide, and any combination thereof; (ii) said cell is a prokaryote cell or a eukaryote cell; (iii) said cell is a transgenic cell or a cell transfected with a plasmid or expression vector comprising a polynucleotide encoding a polypeptide comprising an amino acid sequence selected from the group consisting of: (1) SEQ ID Nos: 1-12 and 14-16, or a functional analog having at least 88% sequence homology thereto; and (2) SEQ ID No 13, or a functional analog having at least 92% sequence homology thereto; (iv) further comprising a step preceding step (a), comprising introducing or transfecting said cell with said plasmid or expression vector; and (v) any combination of (i) to (iv).

28. (canceled)

29. (canceled)

30. (canceled)

31. A method for producing D-psicose comprising the steps:
a. contacting the composition of claim 18 with an effective amount of fructose, thereby obtaining a reaction mixture; and
b. subjecting said reaction mixture to conditions suitable for at least partial conversion of said fructose to D-psicose,
thereby producing D-psicose.

32. The method of claim 31, wherein said conditions comprise: (i) a temperature between 25° C. and 75° C.; (ii) a pH of between 3 and 8.5, or both (i) and (ii).

33. The method of claim 31, wherein said subjecting comprises contacting said reaction mixture with a divalent metal cation, and optionally wherein said divalent cation is selected from the group consisting of: Co²⁺, Mg²⁺, Mn²⁺, Mo²⁺, Ni², and any combination thereof.

34. (canceled)

35. The method of claim 33, wherein said divalent metal cation is present in said reaction mixture in a concentration ranging from 0.1 mM to 10 mM.

36. The method of claim 31, further comprising a step comprising isolating said D-psicose from said reaction mixture.

37. The method of claim 36, wherein said isolating comprises: extraction, precipitation, membrane filtration, or any combination thereof.


SEQUENCE LISTING (32 sequences)

SEQ ID NO: 1 - protein, 287 aa, Artificial Sequence
  Synthetic
MKFAICNETFQPDAPPLWPLEKAFAYARELGYTGIEFAPFDATDVSAERRAEVRELLEKYQLESIGLHWLLAKTELTSPDAAVRDRTSEYLAELARLCRDLGGSVMCGVLGSPQQRNLLPGVTHDQAMEYAADVIRAAMPALEDHGVTLALEPLGPAEGDFLLTAESGIQLAQMIDSPHVRLHLDVKAMSLEAKPIPDIIRDSADYLAHFHANDPNKRGPGMGDVDFVPIFQALQEINYQGWVSVEVFDYEPGVVERLARESISYMQRCLDKLYSSVSGGERPPPDN

SEQ ID NO: 2 - protein, 287 aa, Artificial Sequence
  Synthetic
MKFAICQELFEPDAPPLTDWAEQCRIIAEVGYTGIEVAPFDLASVPAATLTEMRNTAERHGLEIIGLHWLLAKTNLTSPDAAVRTATADYLKLLADTCAALGGRVLAGVFGSPQQRNLLAGVTTEQAMDFAAEVFRAAMPTFAERDVVLCMEPLTPKETDFINTCADAVALMQMVDHPSFVLHQDVKAMLLAESDVPELIHRHKDICGHFHVNDTNLLGPGMGETDYHPILKALLEVGYDGWVSVEVFDYSPGAVEKIAKKSMDYMQQVLSDLYKAVSGVSGPPCDN

SEQ ID NO: 3 - protein, 290 aa, Artificial Sequence
  Synthetic
MKLGIHSFVWTGGSTQEDLEGAMEKSHELGYDLIEFPRLDPKKFDVAWLAKRLEDSGLKVVVTMGLPLDGDISSEDADVVKSGEQILDDAVAVTRDLGGEKLGGILFSAHGKYQSMPTRKGWDNSVAVMRKVADRAKAAGVTLNLEIVNRFESNLLNTTAQGLAFIEDTGSDNIFLHLDTFHMNIEEADPAQAIRHAGDKLGYFHIGESNRGYLGAGTIDFPPIFDALVAIGYEDYVTFESFSSEVVDKDLSITTGIWRNTWTDNVEVARHAKAFIEARYGEARRKAQIA

SEQ ID NO: 4 - protein, 290 aa, Artificial Sequence
  Synthetic
MRLGIHSFVWTGGQTQEGLEMALEKSAEHGYRLIEFAYLRPEKFDLDRLAKKAQSLDLEIAVTMGLPLDADVSSEDADVVARGKAMLADAVKAVRDIGGNKLGGILYSAHTKYNRMPTQRGWKNSVEAIAKTAEIAKACGVDLVLEVVNRFETNLLNTTAQGLKFIEDVGSDDVRLHLDTFHMNIEEADPAAAIRLAGDKLGYFHIGENNRGYLGDGVIDFDAIFDALLDIGYDRDIVFESFSSAVVDEGLSLACAIWRDTWTDNDELAAHAKQFIELKYDEAQRRRATN

SEQ ID NO: 5 - protein, 290 aa, Artificial Sequence
  Synthetic
MRLGIHSFVWTGGQTQSMLEEAMEKSASCGYNLIEFAYLRPEKFDLDALAKRAQALKLDIAVTMGLPFSHDVSSEDADTVKAGEGMLADAVRAVRDIGGSKLGGILYSAHSKYSRMPTKKGWDNSVAAIAKTADLAKSAGVDLVLEVVNRFETNLLNTTAQGLAFIEQVGSEHVRLHLDTFHMNIEEANVAAAIRLAGDKIGYFHIGESNRGYLGDGNINFDLIFDALLDIDYQRDITFESFSGAVVDEGLSLACAIWRDTWTENMPLAQHAKQFIDLKMDEAKRRRATV

SEQ ID NO: 6 - protein, 290 aa, Artificial Sequence
  Synthetic
MRLGIHSFVWTGGQTQEGLEMALEKSAEHGYRCIEFAYLRPELFDLDRLARKSQSLDVQIGVTMGLPLDKDVSSEDPDVVAAGKQMLADAVRAVRDVGGDKLGGILYSAHTKYNRQPTADGWKNSVEAIAETGEIAKAAGVDLVLEVVNRFETNLLNTTAQGLKFIDDTGSDHVRLHLDTFHMQIEEANPAAAIRLAGDKLGYFHIGESNRGYLGDGTIDWDPIFDALLDIGYDRDVVFESFSTAIVDEGLSLACAIWRDTWTENDELAAHAKQFIELKWAEAQRRRATN

SEQ ID NO: 7 - protein, 290 aa, Artificial Sequence
  Synthetic
MRLGIHSFVWTGGQTQEGLEMALEKSAEHGYRTIEFAYLRPELFDLDRLARKAQSLDVEIGVTMGLPLDKDVSSEDRDVVARGKQMLADAVRAVRDIGGNKLGGILYSAHTKYNRLPTADGWKNSVEAIAETGEIAKAAGVDLVLEVVNRFETNLLNTTAQGLKFIDDVGSDHVRLHLDTFHMNIEEANPAAAIRLAGDKLGYFHIGESNRGYLGDGNIDFDLIFDALLDIDYQRDVVFESFSTAIVDEGLSLACAIWRDTWTENDPLAAHAKQFIELKYAEAQRRRATN

SEQ ID NO: 8 - protein, 290 aa, Artificial Sequence
  Synthetic
MRLGIHSFVWTGGQTQEGLEMALNKTAEHGYRTIEFAYLRPEKFNLDRLAKLAQSLDVEIGVTMGLPLDKDVSSEDAAAVAAGKQMLADAVRAVRDIGGNKLGGILYSAHTKYNRQPTKKGWDNSVAAIAATAEIAKEAGVDLVLEVVNRFETNLLNTTAQGLKFISEVGSDHVRLHLDTFHMNIEEANPAAAIRLAGDKIGYFHIGESNRGYLGDGVINFDLIFDALTDIDYKRDIVFESFSTAVVDEGLSLACAIWRDTWTDNDELAAHAKRYIELKYDEAKRRRATN

SEQ ID NO: 9 - protein, 290 aa, Artificial Sequence
  Synthetic
MKLGIHSFVWTGGTTQEDLEGAMEKSHDLGYGLIEFPRLDPKKFDVSWLAKRLEDYGLKVVVTMGLPLDGDISSEDAAVVKRGEQILDDAVAITRDLGGEKLGGIIFSAHTKYQSMPTRKGWDNSVAALSRVADRAKAAGVSLNLEIVNRFESNLLNTTAQGLAFIEETGSDNIFLHLDTFHMNIEEADPAQAIRLAGDKLGYFHIGESNRGYLGSGVINFPAIFDALVAIGYEDWITFESFSSEVVDEDLSITCAIWRNTWTDNVEVARLAKAFIEARYGEARRKAQTA

SEQ ID NO: 10 - protein, 288 aa, Artificial Sequence
  Synthetic
MQIGAHGLVFTGTFDEAGLRRAIEGTKHAGFDLIEIPLMDVDGFDSALAGRMLADNDLAVTASLGLTAATDLTSEDPAVVAAGERMLERCLDHVAAMGGDVLCGVIYSAMQKYMAPATERGIANSQAAIARLAEKAAERGIRLSLEVVNRYESNVFNTGRGALAFLDGVGRDDVSVHLDTYHMNIEESDLFQPVHDVAGRLGYVHIGESHRGYLGTGTVDFGTFFRALHDVRYDGPVVFESFSSAVVSAELSNTLGIWRNLWEDSDDLAAHANRFIRDGLHAVRTIDL

SEQ ID NO: 11 - protein, 288 aa, Artificial Sequence
  Synthetic
MDIGCHGLVWTGSFDAQGIRLAAQKTKEAGFDLIEYPLMDPFTFDTEAAVSALAEHGLKASASLGLSDSTDVTSTDPEVVAAGEALLLRAVDVLADMGGTHLCGVIYSAMKKYMEPVTAEGLENSKRTIGRVADHAADRGVNVSLEVVNRYETNVLNTARQAIRFVADVDRPNLGIHLDTYHMNIEESDMFAPVLDAGEVLHYVHIGESHRGYLGTGSVDFDNFFKALGRIGYDGPITFESFSSAVVAPDLSRMLGIWRNLWTDNADLGAHANRFIRDKLTAVETISL

SEQ ID NO: 12 - protein, 288 aa, Artificial Sequence
  Synthetic
MEYGGHALVWAGDWSPEGARQAISGAARAGYDYIEIALLDPWKVDVAMTKDLLQEYGLRAHASLGLSPTTDVTSTDPAIVAKGDELLRKATDVLHALGGTELCGVIYCALGKYPGPASKENRANSVAAMQRLADYAADKGININLEVVNRYETNIMNTGIEGLKFLEEVNRPNAYLHLDTYHMNIEEDGMEKAVLAAGDRLGYVHIGESHRGYLGSGNVDFDTFFAALKKINYQGPITFESFSSAVVDPALSNALCVWRNLWSDSDDLAKKALKFMKARYSGNSQRSP

SEQ ID NO: 13 - protein, 288 aa, Artificial Sequence
  Synthetic
MEYGGHALVWSGDWTPEGARKAISGAARAGYDYIEIALLDPWKVDVALTKDLLQEYNLRAHASLGLSAATDVTSTDPAIVAKGDELLRKATDVLHAIGGTELCGVIYCALGKYPGPASKENRANSVAAMQRLADYAADKGININLEVVNRYETNIMNTGLEGLAFLDEVNRPNAYLHLDTYHMNIEEDGMEKSVLAAKDRLGYVHIGESHRGYLGTGNVDFDSFFAALKKIDYQGPITFESFSSEVVDPKLSNTLCVWRNLWHDSDDLAKKALRFMKERYPGNPQRSP

SEQ ID NO: 14 - protein, 288 aa, Artificial Sequence
  Synthetic
MKYGGHALVWSGDWTPEGARQAISGAARAGYDYIEIALLDPWKVDVALTKDLLQEFNLRAHASLGLSAATDVTSTDMAIVAKGDELLRKATDVLAAIGGKELCGVIYCALGKYPGPASKENRNNSVAAMQRLADYAADKDININLEVVNRYETNIINTGIEGLAFLDEVDRPNAYLHLDTYHMNIEEDGMEKSVLAAKDRLGYVHIGESHRGYLGTGNVDFDTFFAALKKIGYQGPITFESFSSEVVDPNLSNTLCVWRNLWHDSDDLAKKALQFMKERYSGNPQRSP

SEQ ID NO: 15 - protein, 288 aa, Artificial Sequence
  Synthetic
MKFGIHSMVWVGDWSPEHARHAIASSAEAGYDLIELAAIDPATFDVDLTAKLLAEHGIQASASLGLDAATDVSSEDPDVVAAGRARLRDALALVRDTGGTMLCGVIYSALRKYDAPVTERGLANSQETIAALADEAAASGITIGLEFCNRYETNVLNTTAQTLAFIAAVDRPNVMAHLDTYHMNIEEPSMSEPVLAAAGRLGYVHVGESHRGAPGTGSIPWDEFFGALRECGYDGIVTFESFSSEVVHPSFSNTLAIWRNLWTDNRKLATGALAFMRAHLRAAGGRGA

SEQ ID NO: 16 - protein, 288 aa, Artificial Sequence
  Synthetic
MKLGIHALVWVGGWTPEEAELAISSTARAGYDLIEIPALDPSRIDVASTREMLERHGLQASVSLGLAPDTDINSEDVACVDRGRDVLMRALEVSSGIGSTYLGGVIFGAMTKYSAPTTERARANSVAVIKELAQEARGCGTTIGLEFVNRYESNLLNTAQQTLDYLDLVGEDNVVVHADVYHMNIEETDFRTPILACGDRLGYVHVGESHRGYLGTGSVNFPEFFGALKEVGYAGPITFESFSSSVVDPLLSNTLAIWRNLWSDSTDLATQAHAFISAGLADDGGRGA

SEQ ID NO: 17 - DNA, 861 bp, Artificial Sequence
  Synthetic
atgaagtttgcgatttgtaatgagactttccagccagacgctccgccgttatggcctcttgagaaagcattcgcttatgcgcgtgaacttggttacaccggtatcgaattcgcaccttttgatgcgacagacgtaagtgcagaacggcgggccgaggtgcgcgagcttcttgaaaaataccagctcgaatctattggtttacattggctgttagctaaaacagagctcacttccccggatgcagctgtacgggatcggacttccgagtatctggcggaactggcacgtctttgccgggatcttggcggttccgtgatgtgcggcgtcctgggttcgccacaacagcggaacttactgccgggggtaactcacgaccaggcaatggaatatgcagccgatgtaatccgcgcggcgatgccggccttagaagaccacggggtaactctcgctctcgaaccgttaggcccggcagagggggacttcttgctgactgccgaatcgggtattcagttagctcagatgatcgactccccgcatgtacgtcttcatttggatgttaaagcaatgagtctcgaagcgaagccgattccagatattatccgggattctgcagattacttggcgcacttccatgcaaacgacccaaataagcggggccctggcatgggcgacgttgacttcgtccctatctttcaggctttacaggaaattaactaccaaggctgggtatccgtggaagtatttgattatgaaccaggtgtggttgaacgccttgcccgtgaatccatttcatacatgcaacggtgtctggacaaactttattcctcggtatcgggtggcgaacgcccgcctccagacaac

SEQ ID NO: 18 - DNA, 861 bp, Artificial Sequence
  Synthetic
atgaaatttgcgatttgccaggaactgttcgagccggatgcgccgccgctcacagactgggccgagcaatgtcggatcatcgcggaggtcggctacaccgggattgaggttgctccttttgatttagcttcagtaccggcggcaacacttactgaaatgcggaatacggcagagcgccatggccttgaaattatcgggctgcattggttgctcgcgaagacgaatcttacatctcctgatgccgccgtgcggaccgctacagccgactacttgaagttattagcggatacctgcgccgctctggggggtcgggtgctcgccggggtttttggctcacctcaacaacggaacttgttggcaggcgtaacaaccgaacaagcaatggattttgctgcagaagtatttcgggctgccatgccaacgtttgcggagcgggatgtggtcctctgcatggagccgctcacaccgaaggagactgattttattaacacctgcgctgacgctgttgcgctgatgcaaatggttgaccacccaagtttcgtccttcaccaagatgtaaaagcaatgctgcttgccgagagtgatgtgccagagctgatccaccgtcataaagatatctgcggccattttcacgttaatgacactaatttacttgggccaggtatgggggaaacggactatcatccaattctgaaggcgttactggaagtcggttatgacggttgggtttccgtcgaagtgttcgactattcaccaggggcagtcgagaagattgcgaagaaatcgatggattatatgcagcaagtattgtccgatctctataaggcggtttcaggtgtgtctggtccgccatgcgataac

SEQ ID NO: 19 - DNA, 870 bp, Artificial Sequence
  Synthetic
atgaagttagggattcacagtttcgtatggactggggggtccactcaagaagacttagaaggtgctatggaaaagagccatgaattaggctatgatttaatcgagtttccgcgtctggacccgaagaaattcgatgtagcttggctcgccaaacgcctggaagattctggtctgaaagtagtggtgaccatgggtttaccacttgatggcgatatctcatcagaggacgcagacgtggtgaaatctggtgaacaaattctggatgacgctgtagcggtcacccgtgatttaggtggcgagaaacttggtggtatcttgttttcggcccacgggaagtatcaatcaatgccaacacggaaaggttgggataatagtgttgccgtaatgcggaaagtagcagatcgtgcgaaggcggcgggtgtaacactcaacctggagattgtcaatcggttcgagtcaaacttgttgaatacgacagcacagggtttagcatttatcgaagacacgggttctgataacatttttttacatttagatacgtttcacatgaacattgaggaagctgatccggcacaagccatccgtcatgccggcgacaaacttggttatttccatattggggagtcgaaccgggggtatttaggcgcggggactatcgacttcccgccgattttcgacgcgctggtagcgattggctacgaagactacgtcaccttcgagtcattctcctccgaagtagtcgacaaagatttatctatcacaacgggtatttggcggaatacctggacagataatgtcgaggtggcacgtcacgctaaagcattcattgaggcacggtatggtgaagctcgtcgtaaggcacagatcgcg

SEQ ID NO: 20 - DNA, 870 bp, Artificial Sequence
  Synthetic
atgcggttgggtattcactcgttcgtgtggacgggtggccagacgcaggaggggttagaaatggcgctggagaagtcagcggaacacggctaccggttaatcgagtttgcgtacctgcggccggaaaagttcgacctggatcgtctcgcaaagaaagcccaatccctcgacttagagattgcagtaacaatgggtttacctctcgatgcggatgtatcctctgaagatgcagatgtagtcgctcgggggaaagctatgttagcggacgcggtcaaggctgtacgtgatattgggggcaataaattagggggtattctctattcagcccacaccaaatataatcgtatgccgacgcagcggggctggaagaatagtgtagaagctattgcaaagactgcggagattgccaaggcgtgcggcgttgacttagtgctggaagtggtcaatcggtttgagacaaatttgttgaacacaaccgcgcaaggtcttaagttcattgaagacgtaggctccgacgatgtacgtttacatctcgataccttccacatgaatattgaagaggcagatccggcagcggctattcgtcttgcaggggataagctgggctactttcacattggtgaaaacaaccgggggtacctgggtgacggggttatcgacttcgatgctatcttcgacgcgcttttggacatcggttatgatcgggacatcgtctttgagtctttctccagtgccgtagttgacgaggggctgtcgctggcatgtgcaatttggcgtgatacgtggactgataatgatgaattggcggcacatgcgaagcaattcattgagttaaaatacgatgaagcgcaacgtcgtcgtgctactaat

SEQ ID NO: 21 - DNA, 870 bp, Artificial Sequence
  Synthetic
atgcgcttagggattcactcttttgtctggactggggggcaaacgcaaagtatgttagaagaggcaatggaaaaatcagcaagctgcgggtataatctgattgagttcgcttacctccgccctgagaagtttgacctcgatgcgttagcaaagcgtgcccaggcattaaaattagacattgcggtcaccatggggttacctttctctcacgatgtctcatctgaggacgctgacactgttaaggcaggtgaggggatgctcgcagatgcagtgcgcgccgttcgggatatcggcggttcaaagctcggtggtattttgtactccgctcactccaagtacagccggatgccgacgaagaaagggtgggataattccgtagcggccatcgccaagacggcggacttagctaagtcagccggtgtagatcttgttcttgaggttgttaaccgttttgagactaatctgcttaatacgacagcacagggcttggccttcattgagcaggttgggtctgaacacgtgcgtctccatcttgataccttccacatgaatattgaagaggcgaatgtcgcagccgcgatccgtttagctggtgataagatcgggtacttccacattggtgaaagtaatcggggttatctcggggatggtaatattaactttgaccttatctttgatgctctgctcgatattgactatcaacgtgacatcacatttgagtccttttctggcgcggttgttgatgaggggctgtctctggcttgcgcgatttggcgggacacctggacagaaaatatgcctttagcccagcacgcaaaacagttcatcgatcttaagatggacgaggccaagcgccgtcgtgcaacggtg

SEQ ID NO: 22 - DNA, 870 bp, Artificial Sequence
  Synthetic
atgcgccttggtattcactcttttgtctggacgggtggccagacgcaagaagggttagagatggcgttggagaagagcgctgaacatggttatcggtgtattgagtttgcgtacctccggccagagttattcgatttggatcggttggcgcgtaaatcgcagagtctcgacgtgcaaattggggtcacaatgggtttgccacttgacaaagacgtaagctccgaagaccctgatgtagttgcggcggggaagcagatgctcgccgacgcggtacgcgcagtccgggacgtaggcggtgacaagctgggcggcatcttatatagtgcgcacacaaaatataaccgccaacctacagcggatggttggaaaaattcggttgaagccatcgccgagactggggaaatcgctaaggccgccggtgtagatttggtgttggaagtggtgaaccgctttgaaaccaatctgctcaatacaaccgcgcaggggttaaagttcattgatgatacaggcagcgatcatgtccgtctgcaccttgatacatttcacatgcaaattgaggaagctaatcctgctgcggccattcgtctggctggcgataagctggggtattttcacatcggtgagtccaaccggggctacttgggggacggcactatcgactgggaccctatctttgacgctttgctggatattggttatgatcgtgatgtggttttcgaatcgttcagcaccgctatcgtcgatgaaggtcttagtttggcctgcgcaatttggcgcgatacctggacggaaaacgatgagttggcagcacacgctaagcaattcatcgaactcaagtgggccgaggctcagcgtcggcgcgcgaccaac

SEQ ID NO: 23 - DNA, 870 bp, Artificial Sequence
  Synthetic
atgcgcttaggcattcattctttcgtctggaccgggggtcaaacccaggaggggttggaaatggctttggaaaaatctgcagagcacggctatcggactatcgagtttgcgtacctgcgcccagaattgtttgacttggaccggctggctcgtaaagcgcagtccctggacgtggagatcggggtaacgatgggcttaccgttagataaggatgtgtcttcggaggaccgtgacgttgtagcacggggtaagcaaatgctcgccgatgcagtccgggcagttcgtgacattgggggcaataaattggggggtatcttatactccgcacatactaagtacaatcggctgccgactgcagatgggtggaaaaactcggtggaagcgatcgcggaaactggggagatcgccaaagcggctggcgttgaccttgtgcttgaggtggtcaaccgcttcgagacgaacctcctgaataccaccgcacagggtcttaaattcatcgacgacgtgggtagcgatcacgttcgccttcatttggacacgttccacatgaacatcgaggaagcgaatcctgccgctgcgattcgccttgctggtgataaactcggttatttccatattggcgagtctaaccgcgggtacctcggggatgggaatatcgacttcgacttaatttttgatgctttgctggatattgactaccaacgtgacgttgtctttgaatcatttagtactgcaatcgttgatgaggggcttagtttagcatgcgctatctggcgggatacgtggactgaaaacgatcctctggcggcgcacgcgaaacaattcattgaacttaagtacgctgaggcacagcgccgtcgcgccaccaac

SEQ ID NO: 24 - DNA, 870 bp, Artificial Sequence
  Synthetic
atgcgcttaggcattcattcatttgtatggacgggcggccaaacgcaggaaggcttagaaatggcgctcaacaagactgctgagcacggctatcgcacaattgagttcgcttacctgcgcccagagaaatttaacttagatcggcttgccaagcttgcgcagagccttgatgtcgaaatcggtgtgactatggggctgccgcttgataaggatgtgagcagtgaggatgcagcagccgttgcagctgggaaacaaatgcttgcagatgcggttcgcgctgttcgtgatattgggggcaataaattggggggcattttatactccgcccacaccaagtataaccgccagccgactaaaaaaggctgggacaatagtgtggccgcaatcgcagccacagcggagattgccaaagaggcgggcgtcgatttggtccttgaggtggtgaatcgctttgagacaaacttattaaacactactgcgcagggcctgaagtttattagtgaagtcgggtcggaccatgtacggcttcatctggatacatttcacatgaacatcgaggaggccaacccagcagccgcgattcggttagcgggggataaaattgggtacttccacatcggcgagagcaatcgcggttaccttggcgatggggttattaacttcgatctcatttttgacgcattgaccgatatcgactacaagcgtgatatcgtcttcgaaagtttttcgaccgccgtggtagatgaagggctctctctcgcatgtgcgatttggcgtgacacctggacagataacgatgagctcgccgctcatgcgaagcggtatattgagctcaagtacgatgaagccaaacgtcgccgcgccacaaac

SEQ ID NO: 25 - DNA, 870 bp, Artificial Sequence
  Synthetic
atgaagcttggcattcactcattcgtgtggaccgggggtaccacccaggaagatcttgaaggcgcgatggagaagagccatgatcttggttacggcttgattgaatttccacgtctcgatcctaaaaagtttgacgtgtcctggctcgctaaacgcttggaagattacggcctgaaagtggtagtcactatgggcctccctttagatggggacatcagttctgaggacgccgcggttgtcaaacggggtgagcagatccttgacgacgccgttgccatcactcgtgatctcggcggcgagaaattgggcggcattatcttctccgctcacacgaagtaccaatcaatgcctactcgcaaagggtgggacaacagtgtggctgctctgagccgcgtggctgatcgcgctaaagcagcaggggttagcctgaatcttgaaatcgtgaaccgttttgaatcaaaccttctcaacactactgcccaagggctcgcattcattgaagaaactggctctgataatatctttctccacttggacacctttcacatgaacatcgaggaagctgacccagcccaagctattcgcttagccggtgacaagctgggttacttccacattggggaatcaaatcggggctacttagggtcaggtgtgatcaacttcccggcaattttcgacgcccttgtcgcaattggctacgaggattggattacgttcgagagctttagcagtgaggtggtagatgaagatcttagtattacctgtgctatctggcgcaatacttggactgataacgtagaggtagcccggctcgccaaagcgtttatcgaagcgcgttacggggaagcccgtcgtaaggctcagaccgcg

SEQ ID NO: 26 - DNA, 864 bp, Artificial Sequence
  Synthetic
atgcaaatcggtgcgcatgggttagtcttcaccggtaccttcgatgaggcggggctccgccgcgcgatcgaaggcactaaacacgctggtttcgaccttattgagatcccgctcatggacgtagatggcttcgactctgctttagctggtcggatgttggccgacaacgatctcgcggtcacagcgtctctgggcttgactgcggcaacagatttaacgagcgaagatcctgctgttgtagctgctggggaacggatgttagagcgctgcttagaccacgtcgctgctatgggcggggacgtactctgtggcgtgatttatagcgctatgcaaaaatatatggcacctgcgaccgaacgggggatcgctaatagtcaagctgctattgcacggcttgcggagaaggcggcggagcgcggtattcgtcttagtctcgaagttgttaaccggtacgaatcaaacgtctttaacactgggcgtggtgcgcttgccttcctcgacggtgttggtcgggatgacgtctctgtccatttagacacctatcacatgaatattgaagagagtgatctctttcaaccggtccatgatgttgcaggtcgcttggggtacgttcatattggtgaatcgcatcgcggctacttaggcaccggtactgtggacttcggtactttctttcgtgccctgcatgacgtgcgttatgacgggccagtcgtctttgagtccttttcgtccgcagtggtcagtgcagaactctcaaatacattggggatttggcgcaacctctgggaagacagcgacgatttggcagcacatgctaatcgtttcattcgtgacggccttcacgctgtccgtacaattgatttg

SEQ ID NO: 27 - DNA, 864 bp, Artificial Sequence
  Synthetic
atggacattgggtgtcatggtttagtgtggaccggctcatttgacgctcagggcatccgtctcgcggcccagaaaaccaaggaagccggtttcgatctgattgaatatccacttatggatccgttcacctttgacactgaggccgcggtgagcgcccttgcggagcatggcttgaaagcaagtgcgagcctcggtctgtcggactcaacagatgtgacttctaccgatccggaagtggtcgcggcaggcgaagccttactgctgcgggcggtcgatgttcttgctgatatgggtggtacacacttgtgtggcgtaatctacagtgctatgaaaaagtatatggagcctgtaacagccgaaggcttggagaacagcaagcggactatcggccgtgtcgctgatcatgcagcggaccgtggtgtaaacgtgagtcttgaggtggttaatcggtatgagaccaatgtattgaacactgcgcggcaggcgatccggttcgttgcggacgtagaccggccgaatctcggcattcatttggacacgtaccacatgaacatcgaggagagtgacatgtttgcccctgtcttagacgcgggtgaagtcctccattacgttcatatcggtgaatcgcatcgcggctaccttggcacagggtccgtagatttcgacaattttttcaaagccctcggccgtattggttacgatggtccaatcacattcgagtcgttttcgagcgccgtagttgctccagacctttcgcgtatgttaggcatctggcgtaacttatggaccgacaatgcagatttaggcgcacatgcgaatcggttcatccgtgataagcttaccgcagttgaaactattagcctc

SEQ ID NO: 28 - DNA, 864 bp, Artificial Sequence
  Synthetic
atggagtatgggggccacgcgcttgtatgggcaggcgactggtctccagagggcgcacggcaagctatttcgggtgcggcccgggcaggttatgactacattgagatcgccctcctcgacccttggaaagttgatgtagcgatgaccaaagatctgctccaggaatacggtcttcgtgctcatgcgagcttaggtcttagtccgaccaccgatgtaacgtcaaccgacccagctattgtagcgaagggggatgaactgctgcgtaaggctacagatgttctccatgcactcggggggacggaactttgcggcgttatttactgtgcgttaggtaagtacccagggccagcctcaaaagaaaaccgcgcaaatagcgtagcagcaatgcaacgcttggctgactatgcggctgacaagggcattaatattaatttagaagtagtcaaccggtatgagacaaacattatgaatacagggatcgaaggcttaaaatttctggaggaggtgaaccggccaaacgcgtacctccacttggacacttatcacatgaacattgaagaggatggtatggagaaggctgttttagccgcaggggatcgtctcgggtacgtgcatatcggggagtcccaccgtggctatttgggttctggcaacgtggactttgatacgttcttcgctgcattaaagaagattaactatcagggcccgattacttttgagtctttctcttctgccgtagtcgaccctgcgctgtcaaacgcgctttgcgtctggcgcaatttgtggagcgattccgacgaccttgcgaagaaagcattgaagtttatgaaggctcgttattcggggaactctcaacgcagcccg

SEQ ID NO: 29 - DNA, 864 bp, Artificial Sequence
  Synthetic
atggaatacggcggtcatgccttagtgtggtctggggactggacgccggaaggtgctcgtaaggcgatttccggtgcagcacgggcgggttacgactatattgaaatcgcactccttgatccttggaaagtagatgtcgccttaactaaagatttattgcaagagtacaatctccgtgctcacgcatcgttaggtctctctgcagcaacagacgtcactagtacggaccctgccattgtagccaaaggcgacgagctgttacgcaaggcgacggacgttcttcacgccatcgggggcacagaactctgcggtgtaatctactgtgcactgggcaagtatccagggccagccagtaaagaaaatcgcgcgaatagtgtcgcggctatgcaacggctggcggactacgcagcggacaaggggattaacatcaacctggaggttgtcaatcgctatgagacaaacatcatgaacactggcctggaagggctggcatttctggacgaggtcaatcgtcctaatgcgtatctccacttagatacttatcacatgaacatcgaagaagatggcatggaaaagtcggtactcgccgccaaagatcggctgggttacgtgcacattggcgagtctcaccgcggctatctcgggactggcaatgtcgatttcgattccttctttgctgcattaaaaaagattgactatcagggtccaatcacttttgagagcttctcctcggaggtagttgaccctaagctgagtaatacgttgtgtgtatggcggaacctctggcatgactctgacgatctcgcgaaaaaagcactccggttcatgaaggagcggtatccagggaacccacaacggtcgcct

SEQ ID NO: 30 - DNA, 864 bp, Artificial Sequence
  Synthetic
atgaagtacggtggccatgccttggtatggagcggtgattggacgccagaaggcgctcgccaagccatctcgggggcggctcgcgctgggtacgactatatcgaaatcgcgttactggacccatggaaagtagacgtagctttgaccaaggacctcctgcaggagtttaacttacgtgctcacgcgagtctgggcctgagcgcagccacggatgtcaccagtacagacatggctatcgtggccaagggggatgagctgcttcgcaaagccaccgacgtactggcggcgatcggcgggaaagagctgtgtggggttatttactgtgcccttggcaaatacccaggtcctgcctcgaaagagaatcggaacaacagtgtcgctgcgatgcaacgtcttgcggattatgccgcagataaggacatcaatatcaatttggaggtagttaaccgctacgagacaaacattatcaacacaggtatcgaaggtttagcatttctggatgaggtggatcgcccgaatgcctatcttcacttggacacttaccacatgaatatcgaggaagatgggatggagaagtccgtactcgcggctaaagaccgcctcggctatgttcacatcggcgaatcccatcggggctatcttggcacaggcaatgtagattttgatactttctttgcagccttaaagaaaattgggtatcaagggccaattacatttgagtccttcagttctgaggtcgttgaccctaacctgtcgaatacgctgtgtgtctggcgcaatttgtggcatgactcggatgacctcgcgaaaaaggccttgcaattcatgaaggagcgctactcgggtaacccgcaacggagtccg

SEQ ID NO: 31 - DNA, 864 bp, Artificial Sequence
  Synthetic
atgaagtttgggatccattcaatggtgtgggtgggggactggagtcctgaacacgcgcgccatgccatcgcctcaagtgctgaagcgggctacgatttaatcgagttggctgcaatcgacccggctacgttcgatgtagatctgaccgcgaaacttcttgctgagcacgggattcaagcgtcggcttcccttggtcttgatgcggcgaccgacgtatcaagcgaagacccggatgtggtggccgctgggcgcgctcgcttgcgggacgctttggctctcgtccgggataccggtggcacgatgctttgtggcgttatttactccgctctgcggaagtacgacgcaccggtaactgagcgtggtttagccaattcacaagagaccattgcagcactcgcggacgaggccgcggcttccggtattactatcgggctggagttctgcaaccgttatgaaacgaacgtacttaacacgaccgcccagactttggcgttcatcgcagccgtggatcgccctaacgttatggctcacttagatacctaccacatgaacattgaagaaccgtctatgtctgagcctgttcttgcagcggctggccgccttggttacgtccatgttggtgagtctcaccgcggcgcgcctggtactggtagtatcccatgggacgagttcttcggtgccctccgtgagtgtgggtatgatggtattgtgacttttgagagtttttcttctgaagttgttcaccctagtttttccaacacattggccatctggcggaacctgtggactgacaatcgcaaacttgcaaccggtgctcttgcttttatgcgggcccatttgcgggccgctggcggtcgcggggcc

SEQ ID NO: 32 - DNA, 864 bp, Artificial Sequence
  Synthetic
atgaaactcgggattcatgcattagtgtgggtaggtggttggacacctgaggaagctgaactcgccattagtagcacggcgcgcgcagggtacgatctcattgaaatccctgcgctggacccgtcgcgtattgacgttgcttccacccgggagatgcttgagcgccatggtttgcaagcaagcgtgtccctgggtctcgctcctgacaccgacatcaattcggaagatgtagcgtgtgtagatcgtggccgggatgtcttgatgcgtgcactggaggttagttctggcatcggttccacataccttgggggcgtaattttcggggctatgactaagtacagcgcccctaccacagagcgggcccgcgcaaatagcgtggcggtaatcaaggagttggcccaagaagcccgtggctgcgggacaacgattgggcttgaatttgttaatcggtacgagagtaacttattaaatactgctcaacaaaccctggactacttggatctcgtaggggaagataacgtggtggtacacgctgatgtatatcacatgaacatcgaggagacggatttccgcacaccgattctggcttgcggcgaccgtctgggctatgttcacgtaggcgaatcccaccgtggttacctgggtacaggttcggtaaactttcctgaatttttcggtgctttgaaggaagtcgggtatgcagggcctatcacctttgaatcattctcctccagcgtcgttgaccctctgttgagcaatacattggcgatctggcgtaatctttggagtgacagcacagatctggccactcaggcgcatgcatttatctcggcaggcttggcggatgatggggggcgtggtgcc